(19) United States
(12) Patent Application Publication
(10) Pub. No.: US 2024/0316193 A1
(43) Pub. Date: Sep. 26, 2024

(54) TREATMENT OF CANCER USING A HLA-A2/MAGE-A4 X CD3 BISPECIFIC ANTIBODY AND A 4-1BB (CD137) AGONIST

(71) Applicant: Hoffmann-La Roche Inc., Little Falls, NJ (US)

(72) Inventors: Simon Patrick KEISER, Aarau (CH); Tina WEINZIERL, Eggenwil (CH)

(73) Assignee: Hoffmann-La Roche Inc., Little Falls, NJ (US)

(21) Appl. No.: 18/742,449

(22) Filed: Jun. 13, 2024

Related U.S. Application Data
(63) Continuation of application No. PCT/EP2022/085479, filed on Dec. 13, 2022.

(30) Foreign Application Priority Data
Dec. 14, 2021 (EP) 21214258.2

(51) Int. Cl. A61K 39/395; A61K 38/19; A61P 35/00

(52) U.S. Cl. CPC A61K 39/3955; A61K 38/191; A61K 39/39558; A61P 35/00

(57) ABSTRACT

The present invention relates to the treatment of cancer, in particular to the treatment of cancer using a HLA-A2/MAGE-A4×CD3 bispecific antibody and a 4-1BB (CD137) agonist.

### CROSS-REFERENCE TO RELATED APPLICATIONS

This application is a continuation of Patent Cooperation Treaty Application PCT/EP2022/085479, filed Dec. 13, 2022, which claims priority to European Application No. 21214258.2, filed Dec. 14, 2021, both of which are incorporated herein by reference in their entireties.

### SEQUENCE LISTING

The instant application contains a Sequence Listing which has been submitted electronically in XML format and is hereby incorporated by reference in its entirety. Said XML copy, created on Jun. 13, 2024, is named “P37268-US_Sequence Listing.xml” and is 49,220 bytes in size.

### FIELD OF THE INVENTION

The present invention relates to the treatment of cancer, in particular to the treatment of cancer using a HLA-A2/MAGE-A4×CD3 bispecific antibody and a 4-1BB (CD137) agonist.

### BACKGROUND

T-cell activating bispecific antibodies are a promising class of cancer therapeutics, designed to engage cytotoxic T cells against tumor cells. The simultaneous binding of such an antibody to CD3 on T-cells and to an antigen expressed on the tumor cells will force a temporary interaction between tumor cell and T cell, causing activation of the T-cell and subsequent lysis of the tumor cell.

MAGE-A4 (Melanoma-associated antigen 4) is a member of the MAGE family of Cancer Testis Antigens (CTAs). The MAGE-A family of proteins encompasses 12 highly homologous genes clustered at Xq26-28 and characterized by the presence of a conserved domain (MAGE Homology Domain, MHD). Despite having been discovered more than 20 years ago, biological functions of MAGE proteins still remain poorly understood. Based on their expression pattern, the MAGE family can be divided into two subfamilies, type-I and type-II, with the MAGE-A group belonging to the type-I subfamily whose expression is restricted to germ line and cancer cells. A correlation between overexpression of type-I MAGEs and cancer malignance, tumor growth and poor patient prognosis has been suggested by many reports. Intracellular proteins such as MAGE-A4 can be degraded in the proteasome, processed and presented on the cell surface by major histocompatibility complex (MHC) I as T cell epitopes. As such, MAGE-A4-derived peptides such as MAGE-A4 p230-239 (GVYDGREHTV) are presented in the context of HLA-A2 on the cell surface and can trigger T cell recognition. Given its expression pattern, MAGE-A4 may be a promising target for cancer therapy.

T-cell activating bispecific antibodies targeting HLA-A2/MAGE-A4 have been described in WO 2021/122875. Such T-cell activating bispecific antibodies may be useful, e.g., in the treatment of MAGE-A4-expressing cancers. A HLA-A2/MAGE-A4×CD3 bispecific antibody (“MAGE-A4-TCB”) is currently under investigation in a phase I clinical trial in patients with MAGE-A4-expressing solid tumors, carrying the HLA-A*02:01 allele (NCT05129280).

Unfortunately, treatment of solid tumors with T-cell based immunotherapies still faces many challenges, the most prominent one being overcoming the hostile tumor microenvironment (TME). Fibroblast activation protein (FAP) is highly expressed by cancer-associated fibroblasts (CAFs) and has the ability to modulate the tumor microenvironment (TME) by remodeling the extracellular matrix (ECM). FAP overexpression on CAFs is associated with poor prognosis in various cancers.

In order to maximize the therapeutic benefit of HLA-A2/MAGE-A4-targeting T-cell activating antibodies, particularly in solid tumor cancers, it would thus be desirable to enhance their effect in and despite the presence of the TME.

### BRIEF DESCRIPTION OF THE DRAWINGS

FIG. 1. FIG. 1A shows a schematic illustration of the HLA-A2/MAGE-A4-targeted T-cell bispecific (TCB) antibody molecule used in the Examples (“MAGE-A4 TCB”). The molecule comprises a single antigen binding moiety for CD3, two antigen binding moieties for HLA-A2/MAGE-A4, and an Fc domain. FIG. 1B shows a schematic illustration of the FAP-targeted 4-1BB (CD137) agonist used in the Examples (“FAP-4-1-BBL”). The molecule comprises a 4-1BBL ectodomain trimer, an antigen binding moiety for FAP, and an Fc domain. Black dot: modification in the Fc domain promoting heterodimerization. *: amino acids of opposite charges introduced in the CH and CL domains.

FIGS. 2A-2E show continuous live cell imaging of the HLA-A*02+/MAGE-A4+ tumor cell line SCaBER treated with a combination of MAGE-A4 TCB+FAP-4-1-BBL. SCaBER cells were incubated with human PBMCs at an E:T ratio of 5:1 in the presence of irradiated NIH/3T3-huFAP cells and treated with a combination of 2 nM FAP-4-1-BBL and various concentrations ((A) 50 nM, (B) 8.33 nM, (C) 1.39 nM, (D) 0.23 nM, (E) 0.039 nM) of MAGE-A4 TCB. Tumor cell growth curves (measured as red counts per image) were monitored by continuous live-cell imaging over 120 hours. As controls, SCaBER cells were incubated (in the presence of irradiated NIH/3T3-huFAP cells) with PBMCs in the absence of a treatment (untreated), with 2 nM of FAP-4-1-BBL only (FAP-4-1-BBL) or with various concentrations of MAGE-A4 TCB in absence of FAP-4-1-BBL (MAGE-A4 TCB).

FIGS. 3A-3E. show continuous live cell imaging of the HLA-A*02+/MAGE-A4+ tumor cell line C-33a treated with a combination of MAGE-A4 TCB+FAP-4-1-BBL. C-33a cells were incubated with human PBMCs at an E:T ratio of 5:1 in the presence of irradiated NIH/3T3-huFAP cells and treated with a combination of 2 nM FAP-4-1-BBL and various concentrations ((FIG. 3A) 50 nM, (FIG. 3B) 8.33 nM, (FIG. 3C) 1.39 nM, (FIG. 3D) 0.23 nM, (FIG. 3E) 0.039 nM) of MAGE-A4 TCB. Tumor cell growth curves (measured as red counts per image) were monitored by continuous live-cell imaging over 120 hours. As controls, C-33a cells were incubated (in the presence of irradiated NIH/3T3-huFAP cells) with PBMCs in the absence of a treatment (untreated), with 2 nM of FAP-4-1-BBL only (FAP-4-1-BBL) or with various concentrations of MAGE-A4 TCB in absence of FAP-4-1-BBL (MAGE-A4 TCB).

FIGS. 4A-4G. show quantification of cytokines and granzyme B released by MAGE-A4 TCB-activated T cells. SCaBER cells were incubated with human PBMCs at an E:T ratio of 5:1 in the presence of irradiated NIH/3T3-huFAP cells and treated with a combination of 2 nM FAP-4-1-BBL and various concentrations (50 nM-1 pM) of MAGE-A4 TCB. As controls, SCaBER cells were incubated (in the presence of irradiated NIH/3T3-huFAP cells) with PBMCs in the absence of a treatment (untreated), with 2 nM of FAP-4-1-BBL only (FAP-4-1-BBL) or with various concentrations of MAGE-A4 TCB in absence of FAP-4-1-BBL (MAGE-A4 TCB). Depicted are dose-response curves of cytokine release into the supernatant in pg/mL after 48 hours of incubation as determined by Luminex immunoassays. (FIG. 4A) Interferon (IFN)-γ, (FIG. 4B) interleukin (IL)-2, (FIG. 4C) tumor necrosis factor (TNF)-α, (FIG. 4D) IL-6, (FIG. 4E) IL-8, (FIG. 4F) IL-10, (FIG. 4G) granzyme B.

FIGS. 5A-5G show quantification of cytokines and granzyme B released by MAGE-A4 TCB-activated T cells. C33-a cells were incubated with human PBMCs at an E:T ratio of 5:1 in the presence of irradiated NIH/3T3-huFAP cells and treated with a combination of 2 nM FAP-4-1-BBL and various concentrations (50 nM-1 pM) of MAGE-A4 TCB. As controls, C-33a cells were incubated (in the presence of irradiated NIH/3T3-huFAP cells) with PBMCs in the absence of a treatment (untreated), with 2 nM of FAP-4-1-BBL only (FAP-4-1-BBL) or with various concentrations of MAGE-A4 TCB in absence of FAP-4-1-BBL (MAGE-A4 TCB). Depicted are dose-response curves of cytokine release into the supernatant in pg/mL after 48 hours of incubation as determined by Luminex immunoassays. (FIG. 5A) Interferon (IFN)-γ, (FIG. 5B) interleukin (IL)-2, (FIG. 5C) tumor necrosis factor (TNF)-α, (FIG. 5D) IL-6, (FIG. 5E) IL-8, (FIG. 5F) IL-10, (FIG. 5G) granzyme B.

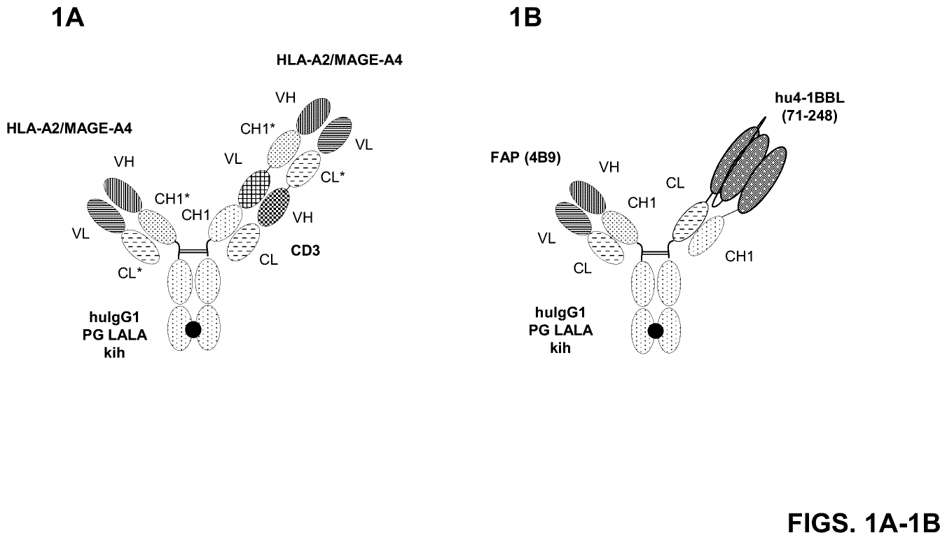

### DESCRIPTION OF THE INVENTION

The present invention enhances T-cell responses in and despite the presence of the TME (e.g. in solid tumor cancers), by a combination treatment of a HLA-A2/MAGE-A4 targeted T-cell activating bispecific antibody with a 4-1BB (CD137) agonist providing a positive costimulatory signal (4-1BBL) to T cells, in particular a 4-1BB (CD137) agonist targeting a stromal antigen such as Fibroblast-activation protein (FAP). As described above, FAP is upregulated on cancer-associated fibroblasts in the tumor, and the FAP specificity of the 4-1BB (CD137) agonist therefore provides T-cell co-stimulation tightly restricted to the tumor.

The present inventors have found that combination of HLA-A2/MAGE-A4 targeted T-cell activating bispecific antibodies with a 4-1BB (CD137) agonist leads to enhanced activity in MAGE-A4-expressing cancers as compared to HLA-A2/MAGE-A4 targeted T-cell activated bispecific antibody alone.

Using human cancer cell lines, the inventors have surprisingly found that tumor cell lysis induced by HLA-A2/MAGE-A4×CD3 bispecific antibody in the presence of FAP-expressing fibroblast cells was enhanced by the addition of the 4-1BB agonist FAP-4-1BBL, even in cell lines and/or at doses where the HLA-A2/MAGE-A4×CD3 bispecific antibody alone did not show activity.

Accordingly, in a first aspect, the present invention provides a HLA-A2/MAGE-A4×CD3 bispecific antibody for use in the treatment of a cancer in an individual, wherein the treatment comprises administration of the HLA-A2/MAGE-A4×CD3 bispecific antibody in combination with a 4-1BB (CD137) agonist.

In a further aspect, the present invention provides a 4-1BB (CD137) agonist for use in the treatment of a cancer in an individual, wherein the treatment comprises administration of the 4-1BB (CD137) agonist in combination with a HLA-A2/MAGE-A4×CD3 bispecific antibody.

In one aspect, the invention provides the use of a HLA-A2/MAGE-A4×CD3 bispecific antibody in the manufacture of a medicament for the treatment of cancer in an individual, wherein the treatment comprises administration of the HLA-A2/MAGE-A4×CD3 bispecific antibody in combination with a 4-1BB (CD137) agonist.

In a further aspect, the invention provides the use of a 4-1BB (CD137) agonist in the manufacture of a medicament for the treatment of cancer in an individual, wherein the treatment comprises administration of the 4-1BB (CD137) agonist in combination with a HLA-A2/MAGE-A4×CD3 bispecific antibody.

In still a further aspect, the invention provides a method for treating cancer in an individual comprising administering to the individual a HLA-A2/MAGE-A4×CD3 bispecific antibody and a 4-1BB (CD137) agonist.

In one aspect, the invention also provides a kit comprising a first medicament comprising a HLA-A2/MAGE-A4×CD3 bispecific antibody and a second medicament comprising a 4-1BB (CD137) agonist, and optionally further comprising a package insert comprising instructions for administration of the first medicament in combination with the second medicament for treating cancer in an individual.

The HLA-A2/MAGE-A4×CD3 bispecific antibodies, 4-1BB (CD137) agonists, methods, uses or kits described above and herein, may incorporate, singly or in combination, any of the features described in the following (unless the context dictates otherwise).

The HLA-A2/MAGE-A4×CD3 bispecific antibody herein is a bispecific antibody that specifically binds to CD3 and to HLA-A2/MAGE-A4, particularly HLA-A2/MAGE-A4ₚ₂₃₀₋₂₃₉. Particularly useful HLA-A2/MAGE-A4×CD3 bispecific antibodies for use in the present invention are described e.g. in PCT publication no. WO 2021/122875 (incorporated herein by reference in its entirety).

The term “bispecific” means that the antibody is able to specifically bind to at least two distinct antigenic determinants. Typically, a bispecific antibody comprises two antigen binding sites, each of which is specific for a different antigenic determinant. In certain aspects, the bispecific antibody is capable of simultaneously binding two antigenic determinants, particularly two antigenic determinants expressed on two distinct cells.

As used herein, the term “antigenic determinant” is synonymous with “antigen” and “epitope”, and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM).

As used herein, the term “antigen binding moiety” refers to a polypeptide molecule that specifically binds to an antigenic determinant. In one aspect, an antigen binding moiety is able to direct the entity to which it is attached (e.g. a second antigen binding moiety) to a target site, for example to a specific type of tumor cell bearing the antigenic determinant. In another aspect an antigen binding moiety is able to activate signaling through its target antigen, for example a T cell receptor complex antigen. Antigen binding moieties include antibodies and fragments thereof as further defined herein. Particular antigen binding moieties include an antigen binding domain of an antibody, comprising an antibody heavy chain variable region and an antibody light chain variable region. In certain aspects, the antigen binding moieties may comprise antibody constant regions as further defined herein and known in the art. Useful heavy chain constant regions include any of the five isotypes: α, δ, ε, γ, or μ. Useful light chain constant regions include any of the two isotypes: κ and λ.

By “specific binding” is meant that the binding is selective for the antigen and can be discriminated from unwanted or non-specific interactions. The ability of an antigen binding moiety to bind to a specific antigenic determinant can be measured either through an enzyme-linked immunosorbent assay (ELISA) or other techniques familiar to one of skill in the art, e.g. surface plasmon resonance (SPR) technique (analyzed e.g. on a BIAcore instrument) (Liljeblad et al., Glyco J 17, 323-329 (2000)), and traditional binding assays (Heeley, Endocr Res 28, 217-229 (2002)). In one aspect, the extent of binding of an antigen binding moiety to an unrelated protein is less than about 10% of the binding of the antigen binding moiety to the antigen as measured, e.g., by SPR. In certain aspects, an antigen binding moiety that binds to the antigen, or an antibody comprising that antigen binding moiety, has a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M).

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., an antigen binding moiety and an antigen, or a receptor and its ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well established methods known in the art, including those described herein. A particular method for measuring affinity is Surface Plasmon Resonance (SPR).

“CD3” refers to any native CD3 from any vertebrate source, including mammals such as primates (e.g. humans), non-human primates (e.g. cynomolgus monkeys) and rodents (e.g. mice and rats), unless otherwise indicated. The term encompasses “full-length,” unprocessed CD3 as well as any form of CD3 that results from processing in the cell. The term also encompasses naturally occurring variants of CD3, e.g., splice variants or allelic variants. In one aspect, CD3 is human CD3, particularly the epsilon subunit of human CD3 (CD3ε). The amino acid sequence of human CD3ε is shown in UniProt (www.uniprot.org) accession no. P07766 (entry version 217), or NCBI (www.ncbi.nlm.nih.gov/) RefSeq NP_000724.1. See also SEQ ID NO: 24. The amino acid sequence of cynomolgus [Macaca fascicularis] CD3ε is shown in NCBI GenBank no. BAB71849.1. See also SEQ ID NO: 25.

“MAGE-A4” stands for “Melanoma-associated antigen 4”, which is a member of the MAGE family of Cancer Testis Antigens (CTAs). The MAGE-A family of proteins encompasses 12 highly homologous genes clustered at Xq26-28 and characterized by the presence of a conserved domain (MAGE Homology Domain, MHD). Human MAGE-A4 is described in UniProt (www.uniprot.org) accession no. P43358 (entry version 174), and an amino acid sequence of human MAGE-A4 is also shown in SEQ ID NO: 21 herein. “MAGE-A4” as used herein, refers to any native MAGE-A4 from any vertebrate source, including mammals such as primates (e.g. humans), non-human primates (e.g. cynomolgus monkeys) and rodents (e.g. mice and rats), unless otherwise indicated. The term encompasses “full-length,” unprocessed MAGE-A4 as well as any form of MAGE-A4 that results from processing in the cell. The term also encompasses naturally occurring variants of MAGE-A4, e.g., splice variants or allelic variants. In one aspect, MAGE-A4 is human MAGE-A4, particularly the protein of SEQ ID NO: 21.

By “MAGE-A4ₚ₂₃₀₋₂₃₉” or “p230-239 peptide” is meant the MAGE-A4 derived peptide having the amino acid sequence GVYDGREHTV (SEQ ID NO: 22; position 230-239 of the MAGE-A4 protein of SEQ ID NO: 21).

“HLA-A2”, “HLA-A*02”, “HLA-A02”, or “HLA-A*2” (used interchangeably) refers to a human leukocyte antigen serotype in the HLA-A serotype group. The HLA-A2 protein (encoded by the respective HLA gene) constitutes the α chain of the respective class I MHC (major histocompatibility complex) protein, which further comprises a β2 microglobulin subunit. A specific HLA-A2 protein is HLA-A201 (also referred to as HLA-A0201, HLA-A02.01, or HLA-A*02:01). In specific aspects, the HLA-A2 protein described herein is HLA-A201. An exemplary sequence of human HLA-A2 is given in SEQ ID NO: 23.

“HLA-A2/MAGE-A4” refers to a complex of a HLA-A2 molecule and a MAGE-A4 derived peptide (also referred to herein as a “MAGE-A4 peptide”), specifically the p230-239 peptide (“HLA-A2/MAGE-A4ₚ₂₃₀₋₂₃₉”).

As used herein, the terms “first”, “second” or “third” with respect to Fab molecules etc., are used for convenience of distinguishing when there is more than one of each type of moiety. Use of these terms is not intended to confer a specific order or orientation of the bispecific antibody unless explicitly so stated.

The term “valent” as used herein denotes the presence of a specified number of antigen binding sites in an antibody. As such, the term “monovalent binding to an antigen” denotes the presence of one (and not more than one) antigen binding site specific for the antigen in the antibody.

The term “antibody” herein is used in the broadest sense and encompasses various antibody structures, including but not limited to monoclonal antibodies, polyclonal antibodies, multispecific antibodies (e.g. bispecific antibodies), and antibody fragments so long as they exhibit the desired antigen-binding activity.

The terms “full length antibody,” “intact antibody,” and “whole antibody” are used herein interchangeably to refer to an antibody having a structure substantially similar to a native antibody structure.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g. scFv), and single-domain antibodies. For a review of certain antibody fragments, see Hudson et al., Nat Med 9, 129-134 (2003). For a review of scFv fragments, see e.g. Plückthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain aspects, a single-domain antibody is a human single-domain antibody (Domantis, Inc., Waltham, MA; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6^{th} ed., W.H. Freeman and Co., page 91 (2007). A single VH or VL domain may be sufficient to confer antigen-binding specificity. As used herein in connection with variable region sequences, “Kabat numbering” refers to the numbering system set forth by Kabat et al., Sequences of Proteins of Immunological Interest, 5th Ed. Public Health Service, National Institutes of Health, Bethesda, MD (1991).

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, MD (1991), referred to as “numbering according to Kabat” or “Kabat numbering” herein. Specifically the Kabat numbering system (see pages 647-660 of Kabat, et al., Sequences of Proteins of Immunological Interest, 5th ed., Public Health Service, National Institutes of Health, Bethesda, MD (1991)) is used for the light chain constant domain CL of kappa and lambda isotype and the Kabat EU index numbering system (see pages 661-723) is used for the heavy chain constant domains (CH1, Hinge, CH2 and CH3), which is herein further clarified by referring to “numbering according to Kabat EU index” in this case.

The term “hypervariable region” or “HVR”, as used herein, refers to each of the regions of an antibody variable domain which are hypervariable in sequence and which determine antigen binding specificity, for example “complementarity determining regions” (“CDRs”). Generally, antibodies comprise six CDRs; three in the VH (HCDR1, HCDR2, HCDR3), and three in the VL (LCDR1, LCDR2, LCDR3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following order in VH (or VL): FR1-H1(L1)-FR2-H2(L2)-FR3-H3(L3)-FR4.

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

A “Fab molecule” refers to a protein consisting of the VH and CH1 domain of the heavy chain (the “Fab heavy chain”) and the VL and CL domain of the light chain (the “Fab light chain”) of an immunoglobulin.

By a “crossover” Fab molecule (also termed “Crossfab”) is meant a Fab molecule wherein the variable domains or the constant domains of the Fab heavy and light chain are exchanged (i.e. replaced by each other), i.e. the crossover Fab molecule comprises a peptide chain composed of the light chain variable domain VL and the heavy chain constant domain 1 CH1 (VL-CH1, in N- to C-terminal direction), and a peptide chain composed of the heavy chain variable domain VH and the light chain constant domain CL (VH-CL, in N- to C-terminal direction). For clarity, in a crossover Fab molecule wherein the variable domains of the Fab light chain and the Fab heavy chain are exchanged, the peptide chain comprising the heavy chain constant domain 1 CH1 is referred to herein as the “heavy chain” of the (crossover) Fab molecule. Conversely, in a crossover Fab molecule wherein the constant domains of the Fab light chain and the Fab heavy chain are exchanged, the peptide chain comprising the heavy chain variable domain VH is referred to herein as the “heavy chain” of the (crossover) Fab molecule.

In contrast thereto, by a “conventional” Fab molecule is meant a Fab molecule in its natural format, i.e. comprising a heavy chain composed of the heavy chain variable and constant domains (VH-CH1, in N- to C-terminal direction), and a light chain composed of the light chain variable and constant domains (VL-CL, in N- to C-terminal direction).

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types, called α (IgA), δ (IgD), ε (IgE), γ (IgG), or μ (IgM), some of which may be further divided into subtypes, e.g. γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (K447), of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991 (see also above). A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g. antigen binding moieties) are not the same. In some aspects the modification promoting association comprises an amino acid mutation in the Fc domain, specifically an amino acid substitution. In a particular aspect, the modification promoting association comprises a separate amino acid mutation, specifically an amino acid substitution, in each of the two subunits of the Fc domain.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. For purposes herein, however, % amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36, and is publicly available from http://fasta.bioch.virginia.edu/fasta_www2/fasta_down.shtml. Alternatively, a public server accessible at http://fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcαRI (CD89).

“Reduced binding”, for example reduced binding to an Fc receptor, refers to a decrease in affinity for the respective interaction, as measured for example by SPR. For clarity, the term includes also reduction of the affinity to zero (or below the detection limit of the analytic method), i.e. complete abolishment of the interaction. Conversely, “increased binding” refers to an increase in binding affinity for the respective interaction.

By “fused” is meant that the components (e.g. a Fab molecule and an Fc domain subunit) are linked by peptide bonds, either directly or via one or more peptide linkers.

The HLA-A2/MAGE-A4×CD3 bispecific antibody comprises a first antigen binding moiety that specifically binds to CD3, and a second antigen binding moiety that specifically binds to HLA-A2/MAGE-A4, particularly HLA-A2/MAGE-A4ₚ₂₃₀₋₂₃₉.

In one aspect, the first antigen binding moiety comprises a heavy chain variable region comprising the heavy chain CDR (HCDR) 1 of SEQ ID NO: 1, the HCDR2 of SEQ ID NO: 2, and the HCDR3 of SEQ ID NO: 3; and a light chain variable region comprising the light chain CDR (LCDR) 1 of SEQ ID NO: 4, the LCDR2 of SEQ ID NO: 5 and the LCDR3 of SEQ ID NO: 6.

In one aspect, the second antigen binding moiety comprises a heavy chain variable region comprising the heavy chain CDR (HCDR) 1 of SEQ ID NO: 9, the HCDR2 of SEQ ID NO: 10, and the HCDR3 of SEQ ID NO: 11; and a light chain variable region comprising the light chain CDR (LCDR) 1 of SEQ ID NO: 12, the LCDR2 of SEQ ID NO: 13 and the LCDR3 of SEQ ID NO: 14.

In a particular aspect, the HLA-A2/MAGE-A4×CD3 bispecific antibody comprises
(i) a first antigen binding moiety that specifically binds to CD3 and comprises a heavy chain variable region comprising the heavy chain CDR (HCDR) 1 of SEQ ID NO: 1, the HCDR2 of SEQ ID NO: 2, and the HCDR3 of SEQ ID NO: 3; and a light chain variable region comprising the light chain CDR (LCDR) 1 of SEQ ID NO: 4, the LCDR2 of SEQ ID NO: 5 and the LCDR3 of SEQ ID NO: 6; and
(ii) a second antigen binding moiety that specifically binds to HLA-A2/MAGE-A4 and comprises a heavy chain variable region comprising the heavy chain CDR (HCDR) 1 of SEQ ID NO: 9, the HCDR2 of SEQ ID NO: 10, and the HCDR3 of SEQ ID NO: 11; and a light chain variable region comprising the light chain CDR (LCDR) 1 of SEQ ID NO: 12, the LCDR2 of SEQ ID NO: 13 and the LCDR3 of SEQ ID NO: 14.

In one aspect, the first antigen binding moiety comprises a heavy chain variable region sequence that is at least about 95%, 96%, 97%, 98%, 99% or 100% identical to the amino acid sequence of SEQ ID NO: 7 and a light chain variable region sequence that is at least about 95%, 96%, 97%, 98%, 99% or 100% identical to the amino acid sequence of SEQ ID NO: 8.

In one aspect, the first antigen binding moiety comprises the heavy chain variable region sequence of SEQ ID NO: 7 and the light chain variable region sequence of SEQ ID NO: 8.

In one aspect, the second antigen binding moiety comprises a heavy chain variable region sequence that is at least about 95%, 96%, 97%, 98%, 99% or 100% identical to the amino acid sequence of SEQ ID NO: 15 and a light chain variable region sequence that is at least about 95%, 96%, 97%, 98%, 99% or 100% identical to the amino acid sequence of SEQ ID NO: 16.

In one aspect, the second antigen binding moiety comprises the heavy chain variable region sequence of SEQ ID NO: 15 and the light chain variable region sequence of SEQ ID NO: 16.

In some aspects, the first and/or the second antigen binding moiety is a Fab molecule. In some aspects, the first antigen binding moiety is a crossover Fab molecule wherein either the variable or the constant regions of the Fab light chain and the Fab heavy chain are exchanged. In such aspects, the second antigen binding moiety preferably is a conventional Fab molecule.

In some aspects wherein the first and the second antigen binding moiety of the bispecific antibody are both Fab molecules, and in one of the antigen binding moieties (particularly the first antigen binding moiety) the variable domains VL and VH of the Fab light chain and the Fab heavy chain are replaced by each other,
i) in the constant domain CL of the first antigen binding moiety the amino acid at position 124 is substituted by a positively charged amino acid (numbering according to Kabat), and wherein in the constant domain CH1 of the first antigen binding moiety the amino acid at position 147 or the amino acid at position 213 is substituted by a negatively charged amino acid (numbering according to Kabat EU index); or
ii) in the constant domain CL of the second antigen binding moiety the amino acid at position 124 is substituted by a positively charged amino acid (numbering according to Kabat), and wherein in the constant domain CH1 of the second antigen binding moiety the amino acid at position 147 or the amino acid at position 213 is substituted by a negatively charged amino acid (numbering according to Kabat EU index).

The bispecific antibody does not comprise both modifications mentioned under i) and ii). The constant domains CL and CH1 of the antigen binding moiety having the VH/VL exchange are not replaced by each other (i.e. remain unexchanged).

In a more specific aspect,
i) in the constant domain CL of the first antigen binding moiety the amino acid at position 124 is substituted independently by lysine (K), arginine (R) or histidine (H) (numbering according to Kabat), and in the constant domain CH1 of the first antigen binding moiety the amino acid at position 147 or the amino acid at position 213 is substituted independently by glutamic acid (E), or aspartic acid (D) (numbering according to Kabat EU index); or
ii) in the constant domain CL of the second antigen binding moiety the amino acid at position 124 is substituted independently by lysine (K), arginine (R) or histidine (H) (numbering according to Kabat), and in the constant domain CH1 of the second antigen binding moiety the amino acid at position 147 or the amino acid at position 213 is substituted independently by glutamic acid (E), or aspartic acid (D) (numbering according to Kabat EU index).

In one such aspect, in the constant domain CL of the second antigen binding moiety the amino acid at position 124 is substituted independently by lysine (K), arginine (R) or histidine (H) (numbering according to Kabat), and in the constant domain CH1 of the second antigen binding moiety the amino acid at position 147 or the amino acid at position 213 is substituted independently by glutamic acid (E), or aspartic acid (D) (numbering according to Kabat EU index).

In a further aspect, in the constant domain CL of the second antigen binding moiety the amino acid at position 124 is substituted independently by lysine (K), arginine (R) or histidine (H) (numbering according to Kabat), and in the constant domain CH1 of the second antigen binding moiety the amino acid at position 147 is substituted independently by glutamic acid (E), or aspartic acid (D) (numbering according to Kabat EU index).

In preferred aspects, in the constant domain CL of the second antigen binding moiety the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the constant domain CH1 of the second antigen binding moiety the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In one aspect, in the constant domain CL of the second antigen binding moiety the amino acid at position 124 is substituted by lysine (K) (numbering according to Kabat) and the amino acid at position 123 is substituted by lysine (K) (numbering according to Kabat), and in the constant domain CH1 of the second antigen binding moiety the amino acid at position 147 is substituted by glutamic acid (E) (numbering according to Kabat EU index) and the amino acid at position 213 is substituted by glutamic acid (E) (numbering according to Kabat EU index).

In one aspect, in the constant domain CL of the second antigen binding moiety the amino acid at position 124 is substituted by lysine (K) (numbering according to Kabat) and the amino acid at position 123 is substituted by arginine (R) (numbering according to Kabat), and in the constant domain CH1 of the second antigen binding moiety the amino acid at position 147 is substituted by glutamic acid (E) (numbering according to Kabat EU index) and the amino acid at position 213 is substituted by glutamic acid (E) (numbering according to Kabat EU index).

In particular aspects, if amino acid substitutions according to the above aspects are made in the constant domain CL and the constant domain CH1 of the second antigen binding moiety, the constant domain CL of the second antigen binding moiety is of kappa isotype.

In some aspects, the first and the second antigen binding moiety are fused to each other, optionally via a peptide linker.

In some aspects, the first and the second antigen binding moiety are each a Fab molecule and either (i) the second antigen binding moiety is fused at the C-terminus of the Fab heavy chain to the N-terminus of the Fab heavy chain of the first antigen binding moiety, or (ii) the first antigen binding moiety is fused at the C-terminus of the Fab heavy chain to the N-terminus of the Fab heavy chain of the second antigen binding moiety.

In some aspects, the HLA-A2/MAGE-A4×CD3 bispecific antibody provides monovalent binding to CD3.

In particular aspects, the HLA-A2/MAGE-A4×CD3 bispecific antibody comprises a single antigen binding moiety that specifically binds to CD3, and two antigen binding moieties that specifically bind to HLA-A2/MAGE-A4. Thus, in some aspects, the HLA-A2/MAGE-A4×CD3 bispecific antibody comprises a third antigen binding moiety, particularly a Fab molecule, more particularly a conventional Fab molecule, that specifically binds to HLA-A2/MAGE-A4. The third antigen binding moiety may incorporate, singly or in combination, all of the features described hereinabove in relation to the second antigen binding moiety (e.g. the CDR sequences, variable region sequences, and/or amino acid substitutions in the constant regions). In some aspects, the third antigen moiety is identical to the first antigen binding moiety (e.g. is also a conventional Fab molecule and comprises the same amino acid sequences).

In particular aspects, the HLA-A2/MAGE-A4×CD3 bispecific antibody further comprises an Fc domain composed of a first and a second subunit. In one aspect, the Fc domain is an IgG Fc domain. In a particular aspect, the Fc domain is an IgG₁ Fc domain. In another aspect the Fc domain is an IgG₄ Fc domain. In a more specific aspect, the Fc domain is an IgG₄ Fc domain comprising an amino acid substitution at position S228 (Kabat EU index numbering), particularly the amino acid substitution S228P. This amino acid substitution reduces in vivo Fab arm exchange of IgG₄ antibodies (see Stubenrauch et al., Drug Metabolism and Disposition 38, 84-91 (2010)). In a further particular aspect, the Fc domain is a human Fc domain. In a particularly preferred aspect, the Fc domain is a human IgG₁ Fc domain. An exemplary sequence of a human IgG₁ Fc region is given in SEQ ID NO: 26.

In some aspects wherein the first, the second and, where present, the third antigen binding moiety are each a Fab molecule, (a) either (i) the second antigen binding moiety is fused at the C-terminus of the Fab heavy chain to the N-terminus of the Fab heavy chain of the first antigen binding moiety and the first antigen binding moiety is fused at the C-terminus of the Fab heavy chain to the N-terminus of the first subunit of the Fc domain, or (ii) the first antigen binding moiety is fused at the C-terminus of the Fab heavy chain to the N-terminus of the Fab heavy chain of the second antigen binding moiety and the second antigen binding moiety is fused at the C-terminus of the Fab heavy chain to the N-terminus of the first subunit of the Fc domain; and (b) the third antigen binding moiety, where present, is fused at the C-terminus of the Fab heavy chain to the N-terminus of the second subunit of the Fc domain.

In particular aspects, the Fc domain comprises a modification promoting the association of the first and the second subunit of the Fc domain. The site of most extensive protein-protein interaction between the two subunits of a human IgG Fc domain is in the CH3 domain. Thus, in one aspect said modification is in the CH3 domain of the Fc domain.

In a specific aspect said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain. The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in some aspects, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine (S), threonine (T), and valine (V). The protuberance and cavity can be made by altering the nucleic acid encoding the polypeptides, e.g. by site-specific mutagenesis, or by peptide synthesis.

In a specific such aspect, in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In a further aspect, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numbering according to Kabat EU index). In a preferred aspect, the first subunit of the Fc domain comprises the amino acid substitutions S354C and T366W, and the second subunit of the Fc domain comprises the amino acid substitutions Y349C, T366S, L368A and Y407V (numbering according to Kabat EU index).

In some aspects, the Fc domain comprises one or more amino acid substitution that reduces binding to an Fc receptor and/or effector function.

In a particular aspect the Fc receptor is an Fcγ receptor. In one aspect the Fc receptor is a human Fc receptor. In one aspect the Fc receptor is an activating Fc receptor. In a specific aspect the Fc receptor is an activating human Fcγ receptor, more specifically human FcγRIIIa, FcγRI or FcγRIIa, most specifically human FcγRIIIa. In one aspect the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular aspect, the effector function is ADCC.

Typically, the same one or more amino acid substitution is present in each of the two subunits of the Fc domain. In one aspect, the one or more amino acid substitution reduces the binding affinity of the Fc domain to an Fc receptor. In one aspect, the one or more amino acid substitution reduces the binding affinity of the Fc domain to an Fc receptor by at least 2-fold, at least 5-fold, or at least 10-fold.

In one aspect, the Fc domain comprises an amino acid substitution at a position selected from the group of E233, L234, L235, N297, P331 and P329 (numberings according to Kabat EU index). In a more specific aspect, the Fc domain comprises an amino acid substitution at a position selected from the group of L234, L235 and P329 (numberings according to Kabat EU index). In some aspects, the Fc domain comprises the amino acid substitutions L234A and L235A (numberings according to Kabat EU index). In one such aspect, the Fc domain is an IgG₁ Fc domain, particularly a human IgG₁ Fc domain. In one aspect, the Fc domain comprises an amino acid substitution at position P329. In a more specific aspect, the amino acid substitution is P329A or P329G, particularly P329G (numberings according to Kabat EU index). In one aspect, the Fc domain comprises an amino acid substitution at position P329 and a further amino acid substitution at a position selected from E233, L234, L235, N297 and P331 (numberings according to Kabat EU index). In a more specific aspect, the further amino acid substitution is E233P, L234A, L235A, L235E, N297A, N297D or P331S. In particular aspects, the Fc domain comprises amino acid substitutions at positions P329, L234 and L235 (numberings according to Kabat EU index). In more particular aspects, the Fc domain comprises the amino acid mutations L234A, L235A and P329G (“P329G LALA”, “PGLALA” or “LALAPG”). Specifically, in preferred aspects, each subunit of the Fc domain comprises the amino acid substitutions L234A, L235A and P329G (Kabat EU index numbering), i.e. in each of the first and the second subunit of the Fc domain the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index). In one such aspect, the Fc domain is an IgG₁ Fc domain, particularly a human IgG₁ Fc domain.

In preferred aspects, the HLA-A2/MAGE-A4×CD3 bispecific antibody comprises
(i) a first antigen binding moiety that specifically binds to CD3, comprising a heavy chain variable region comprising the heavy chain CDR (HCDR) 1 of SEQ ID NO: 1, the HCDR2 of SEQ ID NO: 2, and the HCDR3 of SEQ ID NO: 3; and a light chain variable region comprising the light chain CDR (LCDR) 1 of SEQ ID NO: 4, the LCDR2 of SEQ ID NO: 5 and the LCDR3 of SEQ ID NO: 6, wherein the first antigen binding moiety is a crossover Fab molecule wherein either the variable or the constant regions, particularly the variable regions, of the Fab light chain and the Fab heavy chain are exchanged;
(ii) a second and a third antigen binding moiety that specifically bind to HLA-A2/MAGE-A4, comprising a heavy chain variable region comprising the heavy chain CDR (HCDR) 1 of SEQ ID NO: 9, the HCDR2 of SEQ ID NO: 10, and the HCDR3 of SEQ ID NO: 11; and a light chain variable region comprising the light chain CDR (LCDR) 1 of SEQ ID NO: 12, the LCDR2 of SEQ ID NO: 13 and the LCDR3 of SEQ ID NO: 14, wherein the second and third antigen binding moiety are each a Fab molecule, particularly a conventional Fab molecule;
(iii) an Fc domain composed of a first and a second subunit,

wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain.

In one aspect, the first antigen binding moiety comprises a heavy chain variable region sequence that is at least about 95%, 96%, 97%, 98%, 99% or 100% identical to the amino acid sequence of SEQ ID NO: 7 and a light chain variable region sequence that is at least about 95%, 96%, 97%, 98%, 99% or 100% identical to the amino acid sequence of SEQ ID NO: 8.

In one aspect, the first antigen binding moiety comprises the heavy chain variable region sequence of SEQ ID NO: 7 and the light chain variable region sequence of SEQ ID NO: 8.

In one aspect, the second and third antigen binding moiety comprise a heavy chain variable region sequence that is at least about 95%, 96%, 97%, 98%, 99% or 100% identical to the amino acid sequence of SEQ ID NO: 15 and a light chain variable region sequence that is at least about 95%, 96%, 97%, 98%, 99% or 100% identical to the amino acid sequence of SEQ ID NO: 16.

In one aspect, the second and third antigen binding moieties comprise the heavy chain variable region of SEQ ID NO: 15 and the light chain variable region of SEQ ID NO: 16.

The Fc domain according to the above aspects may incorporate, singly or in combination, all of the features described hereinabove in relation to Fc domains.

In one aspect, the antigen binding moieties and the Fc region are fused to each other by peptide linkers, particularly by peptide linkers as in SEQ ID NO: 18 and SEQ ID NO: 20.

In one aspect, in the constant domain CL of the second and the third Fab molecule under (ii) the amino acid at position 124 is substituted by lysine (K) (numbering according to Kabat) and the amino acid at position 123 is substituted by lysine (K) or arginine (R), particularly by arginine (R) (numbering according to Kabat), and in the constant domain CH1 of the second and the third Fab molecule under (ii) the amino acid at position 147 is substituted by glutamic acid (E) (numbering according to Kabat EU index) and the amino acid at position 213 is substituted by glutamic acid (E) (numbering according to Kabat EU index).

In one aspect, the HLA-A2/MAGE-A4×CD3 bispecific antibody comprises a polypeptide (particularly two polypeptides) comprising a sequence that is at least 80%, 85%, 90%, 95%, 96%, 97%, 98%, or 99% identical to the sequence of SEQ ID NO: 17, a polypeptide comprising a sequence that is at least 80%, 85%, 90%, 95%, 96%, 97%, 98%, or 99% identical to the sequence of SEQ ID NO: 18, a polypeptide comprising a sequence that is at least 80%, 85%, 90%, 95%, 96%, 97%, 98%, or 99% identical to the sequence of SEQ ID NO: 19, and a polypeptide comprising a sequence that is at least 80%, 85%, 90%, 95%, 96%, 97%, 98%, or 99% identical to the sequence of SEQ ID NO: 20.

In one aspect, the HLA-A2/MAGE-A4×CD3 bispecific antibody comprises a polypeptide (particularly two polypeptides) comprising the sequence of SEQ ID NO: 17, a polypeptide comprising the sequence of SEQ ID NO: 18, a polypeptide comprising the sequence of SEQ ID NO: 19, and a polypeptide comprising the sequence of SEQ ID NO: 20.

The HLA-A2/MAGE-A4×CD3 bispecific antibody herein is used in combination with a 4-1BB (CD137) agonist.

The term “4-1BB” or “CD137”, as used herein, refers to any native 4-1BB from any vertebrate source, including mammals such as primates (e.g. humans) and rodents (e.g., mice and rats), unless otherwise indicated. The term encompasses “full-length,” unprocessed 4-1BB as well as any form of 4-1BB that results from processing in the cell. The term also encompasses naturally occurring variants of 4-1BB, e.g., splice variants or allelic variants. The amino acid sequence of an human 4-1BB is shown in UniProt accession no. Q07011 (entry version 185).

“4-1BBL” or “4-1BB ligand” or “CD137L” is a costimulatory TNF ligand family member, which is able to costimulate proliferation and cytokine production of T-cells. Costimulatory TNF family ligands can costimulate TCR signals upon interaction with their corresponding TNF receptors and the interaction with their receptors leads to recruitment of TNFR-associated factors (TRAF), which initiate signalling cascades that result in T-cell activation. 4-1BBL is a type II transmembrane protein. Complete or full length 4-1BBL having the amino acid sequence shown in UniProt accession no. P41273 (entry version 153) has been described to form trimers on the surface of cells. The formation of trimers is enabled by specific motives of the ectodomain of 4-1BBL. Said motives are designated herein as “trimerization region”. The amino acids 50-254 of the human 4-1BBL sequence (SEQ ID NO: 27) form the ectodomain of 4-1BBL, but even fragments thereof are able to form the trimers.

An “ectodomain” is the domain of a membrane protein that extends into the extracellular space (i.e. the space outside the cell), also referred to as “extracellular domain”. The ectodomain of 4-1BBL as defined herein refers to the part of the 4-1BBL protein, particularly the human 4-1BBL protein (UniProt accession no. P41273 (entry version 153)) that extends into the extracellular space (the extracellular domain), but also includes shorter parts or fragments thereof that are responsible for the trimerization and for the binding to the corresponding receptor 4-1BB.

The term “ectodomain of 4-1BBL or a fragment thereof” thus refers to the extracellular domain domain of 4-1BBL, or to parts thereof that are able to bind to 4-1BB and are capable of trimerization. In specific aspects of the invention, the term “ectodomain of 4-1BBL or a fragment thereof” refers to a polypeptide having an amino acid sequence selected from SEQ ID NO: 31 (amino acids 52-254 of human 4-1BBL), SEQ ID NO: 28 (amino acids 71-254 of human 4-1BBL), SEQ ID NO: 30 (amino acids 80-254 of human 4-1BBL), SEQ ID NO: 29 (amino acids 85-254 of human 4-1BBL), SEQ ID NO: 32 (amino acids 71-248 of human 4-1BBL), SEQ ID NO: 33 (amino acids 85-248 of human 4-1BBL), SEQ ID NO: 34 (amino acids 80-248 of human 4-1BBL) and SEQ ID NO: 35 (amino acids 52-248 of human 4-1BBL).

As used herein, the term “antigen binding molecule” refers in its broadest sense to a molecule that specifically binds an antigenic determinant. Examples of antigen binding molecules are antibodies, antibody fragments and scaffold antigen binding proteins.

“Fibroblast activation protein (FAP)”, also known as Prolyl endopeptidase FAP or Seprase (EC 3.4.21), refers to any native FAP from any vertebrate source, including mammals such as primates (e.g. humans) non-human primates (e.g. cynomolgus monkeys) and rodents (e.g. mice and rats), unless otherwise indicated. The term encompasses “full-length,” unprocessed FAP as well as any form of FAP which results from processing in the cell. The term also encompasses naturally occurring variants of FAP, e.g., splice variants or allelic variants. The amino acid sequence of human FAP is shown in UniProt (www.uniprot.org) accession no. Q12884 (entry version 197). The extracellular domain (ECD) of human FAP extends from amino acid position 26 to 760. An antigen binding moiety that binds to FAP as used herein preferably binds to the extracellular domain of FAP. Exemplary anti-FAP binding molecules are described e.g. in PCT publication no. WO 2012/020006.

Particularly useful 4-1BB (CD137) agonists for use in the present invention are described e.g. in PCT publication no. WO 2016/075278 or in PCT publication no. WO 2016/156291 (incorporated herein by reference in their entirety).

In one aspect, the 4-1BB (CD137) agonist comprises 4-1BBL (in particular human 4-1BBL) or a fragment thereof, particularly an ectodomain of 4-1BBL or fragment thereof. In one aspect, the 4-1BB (CD137) agonist comprises three ectodomains of 4-1BBL or fragments thereof (i.e. a first, a second and a third ectodomain of 4-1BBL or fragment thereof). In one aspect, the ectodomain of 4-1BBL or fragment thereof comprises an amino acid sequence that is at least about 95%, 96%, 97%, 98%, 99% or 100% identical to an amino acid sequence selected from the group consisting of SEQ ID NO: 28, SEQ ID NO: 29, SEQ ID NO: 30, SEQ ID NO: 31, SEQ ID NO: 32, SEQ ID NO: 33, SEQ ID NO: 34 and SEQ ID NO: 35. In one aspect, the ectodomain of 4-1BBL of fragment thereof comprises an amino acid sequence that is at least about 95%, 96%, 97%, 98%, 99% or 100% identical to the amino acid sequence of SEQ ID NO: 32. In one aspect, the ectodomain of 4-1BBL or fragment thereof comprises the amino acid sequence of SEQ ID NO: 32. In one aspect, the ectodomain of 4-1BBL or fragment thereof consists of the amino acid sequence of SEQ ID NO: 32.

In a particular aspect, the 4-1BB (CD137) agonist is a molecule comprising three ectodomains of 4-1BBL or fragments thereof, wherein the ectodomains of 4-1BBL or fragments thereof comprise (or consist of) the amino acid sequence of SEQ ID NO: 32.

In one aspect, the 4-1BB (CD137) agonist comprises three ectodomains of 4-1BBL or fragments thereof (i.e. a first, a second and a third ectodomain of 4-1BBL or fragment thereof), wherein the first and the second ectodomain of 4-1BBL or fragment thereof are fused to each other, optionally via a peptide linker (i.e. the first and the second ectodomain of 4-1BBL or fragment thereof are on the same polypeptide), and the third ectodomain of 4-1BBL or fragment thereof is not fused to the first or the second ectodomain of 4-1BBL or fragment thereof (i.e. the third ectodomain of 4-1BBL or fragment thereof is on a separate polypetide from the first and the second ectodomain of 4-1BBL or fragment thereof).

In one aspect, the 4-1BB (CD137) agonist is a molecule comprising a first polypeptide comprising a first and a second ectodomain of 4-1BBL or fragment thereof, and a second polypeptide comprising a third ectodomain of 4-1BBL or fragment thereof. In one aspect, the first and the second ectodomain of 4-1BBL or fragment thereof are fused via a peptide linker, particularly a (G₄S)₂ peptide linker. In one aspect, the first and the second polypeptide are linked by a disulfide bond. In one aspect, the first polypeptide comprises an amino acid sequence that is at least about 95%, 96%, 97%, 98%, 99% or 100% identical to the amino acid sequence of SEQ ID NO: 44. In one aspect, the first polypeptide comprises the amino acid sequence of SEQ ID NO: 44. In one aspect, the second polypeptide comprises an amino acid sequence that is at least about 95%, 96%, 97%, 98%, 99% or 100% identical to the amino acid sequence of of SEQ ID NO: 32. In one aspect, the second polypeptide comprises the amino acid sequence of SEQ ID NO: 32.

In a further aspect, the 4-1BB (CD137) agonist comprises an antigen binding moiety that specifically binds to a tumor-associated antigen, particularly a tumor stromal antigen (i.e. an antigen associated with the tumor stroma), more particularly a tumor fibroblast antigen (i.e. an antigen expressed on cancer-associated fibroblasts).

In a preferred aspect, the antigen binding moiety specifically binds to Fibroblast Activation Protein (FAP), particularly human FAP.

In one aspect, the antigen binding moiety is a Fab molecule, particularly a conventional Fab molecule.

Thus, in a particular aspect, the 4-1BB (CD137) agonist is an antigen binding molecule comprising three ectodomains of 4-1BBL or fragments thereof, and at least one antigen binding moiety that specifically binds to a tumor-associated antigen, in particular an antigen binding moiety that specifically binds to FAP.

In one aspect, the antigen binding moiety that specifically binds to FAP comprises a heavy chain variable region (VH) comprising the heavy chain CDR (HCDR) 1 of SEQ ID NO: 36, the HCDR2 of SEQ ID NO: 37, and the HCDR3 of SEQ ID NO: 38; and a light chain variable region comprising the light chain CDR (LCDR) 1 of SEQ ID NO: 39, the LCDR2 of SEQ ID NO: 40 and the LCDR3 of SEQ ID NO: 41.

In one aspect, the antigen binding moiety that specifically binds to FAP comprises a heavy chain variable region sequence that is at least about 95%, 96%, 97%, 98%, 99% or 100% identical to the amino acid sequence of SEQ ID NO: 42 and a light chain variable region sequence that is at least about 95%, 96%, 97%, 98%, 99% or 100% identical to the amino acid sequence of SEQ ID NO: 43.

In one aspect, the antigen binding moiety that specifically binds to FAP comprises the heavy chain variable region sequence of SEQ ID NO: 42 and the light chain variable region sequence of SEQ ID NO: 43.

In a further aspect, the 4-1BB (CD137) agonist comprises an Fc domain composed of a first and a second subunit.

The Fc domain comprised in the 4-1BB (CD137) agonist may incorporate, singly or in combination, all of the features described hereinabove in relation to Fc domains comprised in the HLA-A2/MAGE-A4×CD3 bispecific antibody.

In particular, in one aspect, the Fc domain comprised in the 4-1BB (CD137) agonist is an IgG Fc domain. In a particular aspect, the Fc domain is an IgG₁ Fc domain. In a further particular aspect, the Fc domain is a human Fc domain. In a particularly preferred aspect, the Fc domain is a human IgG₁ Fc domain.

In a particular aspect, the Fc domain comprises a modification promoting the association of the first and the second subunit of the Fc domain (such as the “knob-into-hole” modification), as described hereinabove in relation to the HLA-A2/MAGE-A4×CD3 bispecific antibody.

In a further particular aspect, the Fc domain comprises one or more amino acid substitution that reduces binding to an Fc receptor and/or effector function (such as the “P329G LALA”, “PGLALA” or “LALAPG” amino acid substitutions), as described hereinabove in relation to the HLA-A2/MAGE-A4×CD3 bispecific antibody.

In a particularly preferred aspect, the Fc domain comprised in the 4-1BB (CD137) agonist is a human IgG₁ Fc domain, wherein each subunit of the Fc domain comprises the amino acid substitutions L234A, L235A and P329G (Kabat EU index numbering).

In one aspect, the 4-1BBL (CD137) agonist is an antigen binding molecule comprising
(i) three ectodomains of 4-1BBL or fragments thereof;
(ii) an antigen binding moiety that specifically binds to FAP, particularly wherein the antigen binding moiety is a Fab molecule; and
(iii) an Fc domain composed of a first and a second subunit, particularly wherein the Fc domain comprises a modification promoting the association of the first and the second subunit of the Fc domain and/or one or more amino acid substitution that reduces binding to an Fc receptor and/or effector function.

In a particular aspect, the 4-1BBL (CD137) agonist is an antigen binding molecule comprising
(i) a first, a second and a third ectodomain of 4-1BBL or fragment thereof;
(ii) an antigen binding moiety that specifically binds to FAP, wherein the antigen binding moiety is a Fab molecule;
(iii) an Fc domain composed of a first and a second subunit, particularly wherein the Fc domain comprises a modification promoting the association of the first and the second subunit of the Fc domain and/or one or more amino acid substitution that reduces binding to an Fc receptor and/or effector function;
(iv) a CL domain and a CH1 domain;

wherein the antigen binding molecule is composed of
(a) a first polypeptide, comprising the (a1) first ectodomain of 4-1BBL or fragment thereof, fused at its C-terminus to the N-terminus of the second ectodomain of 4-1BBL or fragment thereof, (a2) the second ectodomain of 4-1BBL or fragment thereof, fused at its C-terminus to the N-terminus of the CL domain, (a3) the CL domain, fused at its C-terminus to the N-terminus of one of the subunits (e.g. the first subunit) of the Fc domain, and (a4) one of the subunits (e.g. the first subunit) of the Fc domain;
(b) a second polypeptide, comprising (b1) the third ectodomain of 4-1BBL or fragment thereof, fused at its C-terminus to the N-terminus of the CH1 domain, and (b2) the CH1 domain;
(c) a third polypeptide, comprising (c1) the heavy chain of the Fab molecule, fused at its C-terminus to the N-terminus of the other one of the subunits (e.g. the second subunit) of the Fc domain, and (c2) the other one of the subunits (e.g. the second subunit) of the Fc domain; and
(d) a fourth polypeptide, comprising the light chain of the Fab molecule.

Fusion between the various domains of the antigen binding molecule is preferably via peptide linkers, which may also comprise or consist of (part of) an immunoglobulin hinge region. In particular, fusion of the first and the second ectodomain of 4-1BBL or fragment thereof is via a peptide linker, particularly a (G4S)₂ linker.

In one aspect, the first and the second polypeptide are linked to each other by a disulfide bond, particularly a disulfide bond between the CL domain and the CH1 domain.

In one aspect, in the CL domain of the first polypeptide the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the CH1 domain of the second polypeptide the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In one aspect, in the CL domain of the first polypeptide the amino acid at position 124 is substituted by lysine (K) (numbering according to Kabat) and the amino acid at position 123 is substituted by lysine (K) (numbering according to Kabat), and in the CH1 domain of the second polypeptide the amino acid at position 147 is substituted by glutamic acid (E) (numbering according to Kabat EU index) and the amino acid at position 213 is substituted by glutamic acid (E) (numbering according to Kabat EU index).

In a particular aspect, in the CL domain of the first polypeptide the amino acid at position 124 is substituted by lysine (K) (numbering according to Kabat) and the amino acid at position 123 is substituted by arginine (R) (numbering according to Kabat), and in the CH1 domain of the second polypeptide the amino acid at position 147 is substituted by glutamic acid (E) (numbering according to Kabat EU index) and the amino acid at position 213 is substituted by glutamic acid (E) (numbering according to Kabat EU index).

In a particular aspect, if amino acid substitutions according to the above aspects are made in the CL and CH1 domain of the first and second polypeptide, the CL domain of the first polypeptide is of kappa isotype.

In one aspect, the CL domain is a human CL domain, particularly a human CL domain of kappa isotype. In a further aspect, the CH1 domain is a human CH1 domain, particularly a human CH1 domain of y isotype, most particularly a human CH1 domain of γ1 isotype.

In one aspect, the first polypeptide comprises an amino acid sequence that is at least about 95%, 96%, 97%, 98%, 99% or 100% identical to the amino acid sequence of of SEQ ID NO: 45.

In one aspect, the second polypeptide comprises an amino acid sequence that is at least about 95%, 96%, 97%, 98%, 99% or 100% identical to the amino acid sequence of of SEQ ID NO: 46.

In one aspect, the third polypeptide comprises an amino acid sequence that is at least about 95%, 96%, 97%, 98%, 99% or 100% identical to the amino acid sequence of of SEQ ID NO: 47.

In one aspect, the fourth polypeptide comprises an amino acid sequence that is at least about 95%, 96%, 97%, 98%, 99% or 100% identical to the amino acid sequence of of SEQ ID NO: 48.

In one aspect, the 4-1BBL (CD137) agonist is an antigen binding molecule comprising a first polypeptide comprising an amino acid sequence that is at least about 95%, 96%, 97%, 98%, 99% or 100% identical to the amino acid sequence of of SEQ ID NO: 45, a second polypeptide comprising an amino acid sequence that is at least about 95%, 96%, 97%, 98%, 99% or 100% identical to the amino acid sequence of of SEQ ID NO: 46, a third polypeptide comprising an amino acid sequence that is at least about 95%, 96%, 97%, 98%, 99% or 100% identical to the amino acid sequence of of SEQ ID NO: 47, and a fourth polypeptide comprising an amino acid sequence that is at least about 95%, 96%, 97%, 98%, 99% or 100% identical to the amino acid sequence of of SEQ ID NO: 48.

In alternative aspects, the 4-1BB agonist may be an anti-4-1BB antibody, particularly an anti-FAP/anti-4-1BB bispecific antibody.

The term “cancer” refers to the physiological condition in mammals that is typically characterized by unregulated cell proliferation. Examples of cancer include but are not limited to, carcinoma, lymphoma, blastoma, sarcoma and leukemia. More non-limiting examples of cancers include haematological cancer such as leukemia, bladder cancer, brain cancer, head and neck cancer, pancreatic cancer, biliary cancer, thyroid cancer, lung cancer, breast cancer, ovarian cancer, uterine cancer, cervical cancer, endometrial cancer, esophageal cancer, colon cancer, colorectal cancer, rectal cancer, gastric cancer, prostate cancer, skin cancer, squamous cell carcinoma, sarcoma, bone cancer, and kidney cancer. Other cell proliferation disorders include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases.

In some aspects of the HLA-A2/MAGE-A4×CD3 bispecific antibodies, 4-1BB (CD137) agonists, methods, uses and kits of the invention, the cancer is a solid tumor cancer. By a “solid tumor cancer” is meant a malignancy that forms a discrete tumor mass (including also tumor metastasis) located at specific location in the patient's body, such as sarcomas or carcinomas (as opposed to e.g. blood cancers such as leukemia, which generally do not form solid tumors). Non-limiting examples of solid tumor cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, skin cancer, squamous cell carcinoma, bone cancer, liver cancer and kidney cancer. Other solid tumor cancers that are contemplated in the context of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, muscles, spleen, thoracic region, and urogenital system. Also included are pre-cancerous conditions or lesions and cancer metastases.

In one aspect, the cancer is a cancer selected from the group consisting of lung cancer, head and neck cancer, bladder cancer, esophageal cancer, skin cancer, soft tissue cancer, gastric cancer, cervical cancer and ovarian cancer. In one aspect, the cancer is a cancer selected from the group consisting of lung cancer, head and neck cancer, bladder cancer, esophageal cancer, soft tissue cancer and ovarian cancer. In one aspect, the cancer is bladder cancer. In another aspect, the cancer is cervical cancer.

In some aspects, the cancer is a MAGE-A4-positive cancer. By “MAGE-A4-positive cancer” or “MAGE-A4-expressing cancer” is meant a cancer characterized by expression or overexpression of MAGE-A4 in cancer cells. The expression of MAGE-A4 may be determined for example by quantitative real-time PCR (measuring MAGE-A4 mRNA levels), immunohistochemistry (IHC) or western blot assays. In one aspect, the cancer expresses MAGE-A4. In one aspect, the cancer expresses MAGE-A4 in at least 20%, preferably at least 50% or at least 80% of tumor cells as determined by immunohistochemistry (IHC) using an antibody specific for MAGE-A4.

In some aspects, the cancer comprises cells (e.g. fibroblasts) expressing FAP. In some aspects, the cancer expresses FAP, particularly in the tumor stroma.

A “patient”, “subject” or “individual” herein is any single human subject eligible for treatment who is experiencing or has experienced one or more signs, symptoms, or other indicators of cancer. In some aspects, the patient has cancer or has been diagnosed with cancer. The patient may have been previously treated with a HLA-A2/MAGE-A4×CD3 bispecific antibody or another drug, or not so treated. In particular aspects, the patient has not been previously treated with a HLA-A2/MAGE-A4×CD3 bispecific antibody. The patient may have been treated with a therapy comprising one or more drugs other than a HLA-A2/MAGE-A4×CD3 bispecific antibody before the HLA-A2/MAGE-A4×CD3 bispecific antibody therapy is commenced. In particular aspects, the patient carries a HLA-A2 allele, particularly the HLA-A*02:01 allele.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

The HLA-A2/MAGE-A4×CD3 bispecific antibody and the 4-1BB (CD137) agonist are administered in an effective amount.

An “effective amount” of an agent, e.g. a pharmaceutical composition, refers to an amount effective, at dosages and for periods of time necessary, to achieve the desired therapeutic or prophylactic result.

In one aspect, administration of the HLA-A2/MAGE-A4×CD3 bispecific antibody results in activation of T cells, particularly cytotoxic T cells, particularly at the site of the cancer. Said activation may comprise proliferation of T cells, differentiation of T cells, cytokine secretion by T cells, cytotoxic effector molecule release from T cells, cytotoxic activity of T cells, and expression of activation markers by T cells. In one aspect, the administration of the HLA-A2/MAGE-A4×CD3 bispecific antibody results in an increase of T cell, particularly cytotoxic T cell, numbers at the site of the cancer.

In some aspects of the HLA-A2/MAGE-A4×CD3 bispecific antibodies, 4-1BB (CD137) agonists, methods, uses or kits described above and herein, the treatment with or administration of the HLA-A2/MAGE-A4×CD3 bispecific antibody and the 4-1BB (CD137) agonist results in increased activation of T cells, particularly cytotoxic T cells, particularly at the site of the cancer, as compared to treatment with or administration of the HLA-A2/MAGE-A4×CD3 bispecific antibody alone. In particular aspects, the activation comprises cytotoxic activity (specifically lysis of cancer cells) of T cells and/or cytokine (specifically IL-2, TNF-α, and/or interferon-γ) secretion by T cells.

In some aspects of the HLA-A2/MAGE-A4×CD3 bispecific antibodies, 4-1BB (CD137) agonists, methods, uses or kits described above and herein, the treatment with or administration of the HLA-A2/MAGE-A4×CD3 bispecific antibody and the 4-1BB (CD137) agonist results in increased differentiation of naïve T cells towards memory T cells, particularly at the site of the cancer, as compared to treatment with or administration of the HLA-A2/MAGE-A4×CD3 bispecific antibody alone. In one aspect, the differentiation is detected by measurement of CD45RA expression, e.g. using flow cytometry.

In some aspects of the HLA-A2/MAGE-A4×CD3 bispecific antibodies, 4-1BB (CD137) agonists, methods, uses or kits described above and herein, the treatment with or administration of the HLA-A2/MAGE-A4×CD3 bispecific antibody and the 4-1BB (CD137) agonist may result in a response in the individual. In some aspects, the response may be a complete response. In some aspects, the response may be a sustained response after cessation of the treatment. In some aspects, the response may be a complete response that is sustained after cessation of the treatment. In other aspects, the response may be a partial response. In some aspects, the response may be a partial response that is sustained after cessation of the treatment. In some aspects, the treatment with or administration of the HLA-A2/MAGE-A4×CD3 bispecific antibody and the 4-1BB (CD137) agonist may improve the response as compared to treatment with or administration of the HLA-A2/MAGE-A4×CD3 bispecific antibody alone (i.e. without the 4-1BB (CD137) agonist).

In some aspects, the treatment or administration of the HLA-A2/MAGE-A4×CD3 bispecific antibody and the 4-1BB (CD137) agonist may increase response rates in a patient population, as compared to a corresponding patient population treated with the HLA-A2/MAGE-A4×CD3 bispecific antibody alone (i.e. without the 4-1BB (CD137) agonist).

The combination therapy of the invention comprises administration of a HLA-A2/MAGE-A4×CD3 bispecific antibody and a 4-1BB (CD137) agonist.

As used herein, “combination” (and grammatical variations thereof such as “combine” or “combining”) encompasses combinations of a HLA-A2/MAGE-A4×CD3 bispecific antibody and 4-1BB (CD137) agonist according to the invention wherein the HLA-A2/MAGE-A4×CD3 bispecific antibody and the 4-1BB (CD137) agonist are in the same or in different containers, in the same or in different pharmaceutical formulations, administered together or separately, administered simultaneously or sequentially, in any order, and administered by the same or by different routes, provided that the HLA-A2/MAGE-A4×CD3 bispecific antibody and the 4-1BB (CD137) agonist can simultaneously exert their biological effects in the body. For example “combining” a HLA-A2/MAGE-A4×CD3 bispecific antibody and a 4-1BB (CD137) agonist according to the invention may mean first administering the HLA-A2/MAGE-A4×CD3 bispecific antibody in a particular pharmaceutical formulation, followed by administration of the 4-1BB (CD137) agonist in another pharmaceutical formulation, or vice versa.

The HLA-A2/MAGE-A4×CD3 bispecific antibody and the 4-1BB (CD137) agonist may be administered in any suitable manner known in the art. In one aspect, the HLA-A2/MAGE-A4×CD3 bispecific antibody and the 4-1BB (CD137) agonist are administered sequentially (at different times). In another aspect, the HLA-A2/MAGE-A4×CD3 bispecific antibody and the 4-1BB (CD137) agonist are administered concurrently (at the same time). Without wishing to be bound by theory, it may be advantageous to administer the 4-1BB (CD137) agonist prior to and/or concurrently with the HLA-A2/MAGE-A4×CD3 bispecific antibody. In some aspects, the HLA-A2/MAGE-A4×CD3 bispecific antibody is in a separate composition as the 4-1BB (CD137) agonist. In some aspects, the HLA-A2/MAGE-A4×CD3 bispecific antibody is in the same composition as the 4-1BB (CD137) agonist.

The HLA-A2/MAGE-A4×CD3 bispecific antibody and the 4-1BB (CD137) agonist can be administered by any suitable route, and may be administered by the same route of administration or by different routes of administration. In some aspects, the HLA-A2/MAGE-A4×CD3 bispecific antibody is administered intravenously, intramuscularly, subcutaneously, topically, orally, transdermally, intraperitoneally, intraorbitally, by implantation, by inhalation, intrathecally, intraventricularly, or intranasally. In a particular aspect, the HLA-A2/MAGE-A4×CD3 bispecific antibody is administered intravenously. In some aspects, the 4-1BB (CD137) agonist is administered intravenously, intramuscularly, subcutaneously, topically, orally, transdermally, intraperitoneally, intraorbitally, by implantation, by inhalation, intrathecally, intraventricularly, or intranasally. In a particular aspect, the 4-1BB (CD137) agonist is administered intravenously. An effective amount of the HLA-A2/MAGE-A4×CD3 bispecific antibody and the 4-1BB (CD137) agonist may be administered for prevention or treatment of disease. The appropriate route of administration and dosage of the HLA-A2/MAGE-A4×CD3 bispecific antibody and/or the 4-1BB (CD137) agonist may be determined based on the type of disease to be treated, the type of the HLA-A2/MAGE-A4×CD3 bispecific antibody, the type of 4-1BB (CD137) agonist, the severity and course of the disease, the clinical condition of the individual, the individual's clinical history and response to the treatment, and the discretion of the attending physician. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein. The HLA-A2/MAGE-A4×CD3 bispecific antibody and the 4-1BB (CD137) agonist are suitably administered to the patient at one time or over a series of treatments.

Combinations of the invention can be used either alone or together with other agents in a therapy. For instance, a combination of the invention may be co-administered with at least one additional therapeutic agent. In certain aspects, an additional therapeutic agent is an anti-cancer agent, e.g. a chemotherapeutic agent, an inhibitor of tumor cell proliferation, or an activator of tumor cell apoptosis. Combinations of the invention can also be combined with radiation therapy.

A kit as provided herein typically comprises one or more container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a HLA-A2/MAGE-A4×CD3 bispecific antibody to be used in the combinations of the invention. Another active agent is the 4-1BB (CD137) agonist to be used in the combinations of the invention, which may be in the same composition and container like the bispecific antibody, or may be provided in a different composition and container. The label or package insert indicates that the composition(s) is/are used for treating the condition of choice, such as cancer.

In one aspect, the invention provides a kit intended for the treatment of cancer, comprising in the same or in separate containers (a) a HLA-A2/MAGE-A4×CD3 bispecific antibody, and (b) a 4-1BB (CD137) agonist, and optionally further comprising (c) a package insert comprising printed instructions directing the use of the combined treatment as a method for treating cancer. Moreover, the kit may comprise (a) a first container with a composition contained therein, wherein the composition comprises a HLA-A2/MAGE-A4×CD3 bispecific antibody; (b) a second container with a composition contained therein, wherein the composition comprises a 4-1BB (CD137) agonist; and optionally (c) a third container with a composition contained therein, wherein the composition comprises a further cytotoxic or otherwise therapeutic agent. The kit in these aspects of the invention may further comprise a package insert indicating that the compositions can be used to treat cancer. Alternatively, or additionally, the kit may further comprise a third (or fourth) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Amino Acid Sequences

| | | SEQ |
|---|---|---|
| | | ID |
| | Sequence | NO |
| CD3 HCDR1 | GYTMN | 1 |
| CD3 HCDR2 | LINPYKGVSTYNQKFKD | 2 |
| CD3 HCDR3 | SGYYGDSDWYFDV | 3 |
| CD3 LCDR1 | RASQDIRNYLN | 4 |
| CD3 LCDR2 | YTSRLES | 5 |
| CD3 LCDR3 | QQGNTLPWT | 6 |
| CD3 VH | EVQLVESGGGLVQPGGSLRLSCAASGYSFTGYTMNWVRQAPGKGL | 7 |
| | EWVALINPYKGVSTYNQKFKDRFTISVDKSKNTAYLQMNSLRAED | |
| | TAVYYCARSGYYGDSDWYFDVWGQGTLVTVSS | |
| CD3 VL | DIQMTQSPSSLSASVGDRVTITCRASQDIRNYLNWYQQKPGKAPK | 8 |
| | LLIYYTSRLESGVPSRFSGSGSGTDYTLTISSLQPEDFATYYCQQ | |
| | GNTLPWTFGQGTKVEIK | |
| MAGE-A4 HCDR1 | KAMS | 9 |
| MAGE-A4 HCDR2 | SISPSGGSTYYNDNVLG | 10 |
| MAGE-A4 HCDR3 | DVGFFDE | 11 |
| MAGE-A4 LCDR1 | RASQSISSYLA | 12 |
| MAGE-A4 LCDR2 | DASIRDI | 13 |
| MAGE-A4 LCDR3 | QQYSSYPYT | 14 |
| MAGE-A4 VH | AQLVESGGGLVQPGGSLRLSCAASAYFSFKAMSWVRQAPGKGLEW | 15 |
| | VGSISPSGGSTYYNDNVLGRFTISRDNSKNTLYLQMNSLRAEDTA | |
| | VYYCAKDVGFFDEWGQGTLVTVSS | |
| MAGE-A4 VL | DIQMTQSPSSLSASVGDRVTITCRASQSISSYLAWYQQKPGKAPK | 16 |
| | LLIYDASIRDIGVPSRESGSGSGTDETLTISSLQPEDFATYYCQQ | |
| | YSSYPYTFGQGTKLEIK | |
| MAGE-A4 VL- | DIQMTQSPSSLSASVGDRVTITCRASQSISSYLAWYQQKPGKAPK | 17 |
| CL(RK) | LLIYDASIRDIGVPSRFSGSGSGTDFTLTISSLQPEDFATYYCQQ | |
| | YSSYPYTFGQGTKLEIKRTVAAPSVFIFPPSDRKLKSGTASVVCL | |
| | LNNFYPREAKVQWKVDNALQSGNSQESVTEQDSKDSTYSLSSTLT | |
| | LSKADYEKHKVYACEVTHQGLSSPVTKSENRGEC | |
| MAGE-A4 VH- | AQLVESGGGLVQPGGSLRLSCAASAYFSFKAMSWVRQAPGKGLEW | 18 |
| CH1(EE)-Fc(hole, | VGSISPSGGSTYYNDNVLGRFTISRDNSKNTLYLQMNSLRAEDTA | |
| PGLALA) | VYYCAKDVGFFDEWGQGTLVTVSSASTKGPSVFPLAPSSKSTSGG | |
| | TAALGCLVEDYFPEPVTVSWNSGALTSGVHTFPAVLQSSGLYSLS | |
| | SVVTVPSSSLGTQTYICNVNHKPSNTKVDEKVEPKSCDKTHTCPP | |
| | CPAPEAAGGPSVFLFPPKPKDTLMISRTPEVTCVVVDVSHEDPEV | |
| | KFNWYVDGVEVHNAKTKPREEQYNSTYRVVSVLTVLHQDWLNGKE | |
| | YKCKVSNKALGAPIEKTISKAKGQPREPQVCTLPPSRDELTKNQV | |
| | SLSCAVKGFYPSDIAVEWESNGQPENNYKTTPPVLDSDGSFFLVS | |
| | KLTVDKSRWQQGNVFSCSVMHEALHNHYTQKSLSLSP | |
| CD3 VH-CL | EVQLVESGGGLVQPGGSLRLSCAASGYSFTGYTMNWVRQAPGKGL | 19 |
| | EWVALINPYKGVSTYNQKFKDRFTISVDKSKNTAYLQMNSLRAED | |
| | TAVYYCARSGYYGDSDWYFDVWGQGTLVTVSSASVAAPSVFIFPP | |
| | SDEQLKSGTASVVCLLNNFYPREAKVQWKVDNALQSGNSQESVTE | |
| | QDSKDSTYSLSSTLTLSKADYEKHKVYACEVTHQGLSSPVTKSEN | |
| | RGEC | |
| MAGE-A4 VH- | AQLVESGGGLVQPGGSLRLSCAASAYFSFKAMSWVRQAPGKGLEW | 20 |
| CH1(EE)-CD3 VL- | VGSISPSGGSTYYNDNVLGRFTISRDNSKNTLYLQMNSLRAEDTA | |
| CH1-Fc(knob, | VYYCAKDVGFFDEWGQGTLVTVSSASTKGPSVFPLAPSSKSTSGG | |
| PGLALA) | TAALGCLVEDYFPEPVTVSWNSGALTSGVHTFPAVLQSSGLYSLS | |
| | SVVTVPSSSLGTQTYICNVNHKPSNTKVDEKVEPKSCDGGGGSGG | |
| | GGSDIQMTQSPSSLSASVGDRVTITCRASQDIRNYLNWYQQKPGK | |
| | APKLLIYYTSRLESGVPSRFSGSGSGTDYTLTISSLQPEDFATYY | |
| | CQQGNTLPWTFGQGTKVEIKSSASTKGPSVFPLAPSSKSTSGGTA | |
| | ALGCLVKDYFPEPVTVSWNSGALTSGVHTFPAVLQSSGLYSLSSV | |
| | VTVPSSSLGTQTYICNVNHKPSNTKVDKKVEPKSCDKTHTCPPCP | |
| | APEAAGGPSVFLFPPKPKDTLMISRTPEVTCVVVDVSHEDPEVKE | |
| | NWYVDGVEVHNAKTKPREEQYNSTYRVVSVLTVLHQDWLNGKEYK | |
| | CKVSNKALGAPIEKTISKAKGQPREPQVYTLPPCRDELTKNQVSL | |
| | WCLVKGFYPSDIAVEWESNGQPENNYKTTPPVLDSDGSFFLYSKL | |
| | TVDKSRWQQGNVFSCSVMHEALHNHYTQKSLSLSP | |
| Human MAGE-A4 | MSSEQKSQHCKPEEGVEAQEEALGLVGAQAPTTEEQEAAVSSSSP | 21 |
| | LVPGTLEEVPAAESAGPPQSPQGASALPTTISFTCWRQPNEGSSS | |
| | QEEEGPSTSPDAESLFREALSNKVDELAHFLLRKYRAKELVTKAE | |
| | MLERVIKNYKRCFPVIFGKASESLKMIFGIDVKEVDPASNTYTLV | |
| | TCLGLSYDGLLGNNQIFPKTGLLIIVLGTIAMEGDSASEEEIWEE | |
| | LGVMGVYDGREHTVYGEPRKLLTQDWVQENYLEYRQVPGSNPARY | |
| | EFLWGPRALAETSYVKVLEHVVRVNARVRIAYPSLREAALLEEEE | |
| | GV | |
| MAGE-A4ₚ₂₃₀₋₂₃₉ | GVYDGREHTV | 22 |
| HLA-A2 | GSHSMRYFFTSVSRPGRGEPRFIAVGYVDDTQFVREDSDAASQRM | 23 |
| | EPRAPWIEQEGPEYWDGETRKVKAHSQTHRVDLGTLRGYYNQSEA | |
| | GSHTVQRMYGCDVGSDWRFLRGYHQYAYDGKDYIALKEDLRSWTA | |
| | ADMAAQTTKHKWEAAHVAEQLRAYLEGTCVEWLRRYLENGKETLQ | |
| | RTDAPKTHMTHHAVSDHEATLRCWALSFYPAEITLTWQRDGEDQT | |
| | QDTELVETRPAGDGTFQKWAAVVVPSGQEQRYTCHVQHEGLPKPL | |
| | TLRWE | |
| Human CD3 | MQSGTHWRVLGLCLLSVGVWGQDGNEEMGGITQTPYKVSISGTTV | 24 |
| | ILTCPQYPGSEILWQHNDKNIGGDEDDKNIGSDEDHLSLKEFSEL | |
| | EQSGYYVCYPRGSKPEDANFYLYLRARVCENCMEMDVMSVATIVI | |
| | VDICITGGLLLLVYYWSKNRKAKAKPVTRGAGAGGRQRGQNKERP | |
| | PPVPNPDYEPIRKGQRDLYSGLNQRRI | |
| Cynomolgus CD3 | MQSGTRWRVLGLCLLSIGVWGQDGNEEMGSITQTPYQVSISGTTV | 25 |
| | ILTCSQHLGSEAQWQHNGKNKEDSGDRLFLPEFSEMEQSGYYVCY | |
| | PRGSNPEDASHHLYLKARVCENCMEMDVMAVATIVIVDICITLGL | |
| | LLLVYYWSKNRKAKAKPVTRGAGAGGRQRGQNKERPPPVPNPDYE | |
| | PIRKGQQDLYSGLNQRRI | |
| hIgG1 Fc region | DKTHTCPPCPAPELLGGPSVFLFPPKPKDTLMISRTPEVTCVVVD | 26 |
| | VSHEDPEVKFNWYVDGVEVHNAKTKPREEQYNSTYRVVSVLTVLH | |
| | QDWLNGKEYKCKVSNKALPAPIEKTISKAKGQPREPQVYTLPPSR | |
| | DELTKNQVSLTCLVKGFYPSDIAVEWESNGQPENNYKTTPPVLDS | |
| | DGSFFLYSKLTVDKSRWQQGNVFSCSVMHEALHNHYTQKSLSLSP | |
| hu 4-1BBL (50- | ACPWAVSGARASPGSAASPRLREGPELSPDDPAGLLDLRQGMFAQ | 27 |
| 254) | LVAQNVLLIDGPLSWYSDPGLAGVSLTGGLSYKEDTKELVVAKAG | |
| | VYYVFFQLELRRVVAGEGSGSVSLALHLQPLRSAAGAAALALTVD | |
| | LPPASSEARNSAFGFQGRLLHLSAGQRLGVHLHTEARARHAWQLT | |
| | QGATVLGLFRVTPEIPAGLPSPRSE | |
| hu 4-1BBL (71- | REGPELSPDDPAGLLDLRQGMFAQLVAQNVLLIDGPLSWYSDPGL | 28 |
| 254) | AGVSLTGGLSYKEDTKELVVAKAGVYYVFFQLELRRVVAGEGSGS | |
| | VSLALHLQPLRSAAGAAALALTVDLPPASSEARNSAFGFQGRLLH | |
| | LSAGQRLGVHLHTEARARHAWQLTQGATVLGLFRVTPEIPAGLPS | |
| | PRSE | |
| hu 4-1BBL (85- | LDLRQGMFAQLVAQNVLLIDGPLSWYSDPGLAGVSLTGGLSYKED | 29 |
| 254) | TKELVVAKAGVYYVFFQLELRRVVAGEGSGSVSLALHLQPLRSAA | |
| | GAAALALTVDLPPASSEARNSAFGFQGRLLHLSAGQRLGVHLHTE | |
| | ARARHAWQLTQGATVLGLFRVTPEIPAGLPSPRSE | |
| hu 4-1BBL (80- | DPAGLLDLRQGMFAQLVAQNVLLIDGPLSWYSDPGLAGVSLTGGL | 30 |
| 254) | SYKEDTKELVVAKAGVYYVFFQLELRRVVAGEGSGSVSLALHLQP | |
| | LRSAAGAAALALTVDLPPASSEARNSAFGFQGRLLHLSAGQRLGV | |
| | HLHTEARARHAWQLTQGATVLGLFRVTPEIPAGLPSPRSE | |
| hu 4-1BBL (52- | PWAVSGARASPGSAASPRLREGPELSPDDPAGLLDLRQGMFAQLV | 31 |
| 254) | AQNVLLIDGPLSWYSDPGLAGVSLTGGLSYKEDTKELVVAKAGVY | |
| | YVFFQLELRRVVAGEGSGSVSLALHLQPLRSAAGAAALALTVDLP | |
| | PASSEARNSAFGFQGRLLHLSAGQRLGVHLHTEARARHAWQLTQG | |
| | ATVLGLFRVTPEIPAGLPSPRSE | |
| hu 4-1BBL (71- | REGPELSPDDPAGLLDLRQGMFAQLVAQNVLLIDGPLSWYSDPGL | 32 |
| 248) | AGVSLTGGLSYKEDTKELVVAKAGVYYVFFQLELRRVVAGEGSGS | |
| | VSLALHLQPLRSAAGAAALALTVDLPPASSEARNSAFGFQGRLLH | |
| | LSAGQRLGVHLHTEARARHAWQLTQGATVLGLERVTPEIPAGL | |
| hu 4-1BBL (85- | LDLRQGMFAQLVAQNVLLIDGPLSWYSDPGLAGVSLTGGLSYKED | 33 |
| 248) | TKELVVAKAGVYYVFFQLELRRVVAGEGSGSVSLALHLQPLRSAA | |
| | GAAALALTVDLPPASSEARNSAFGFQGRLLHLSAGQRLGVHLHTE | |
| | ARARHAWQLTQGATVLGLFRVTPEIPAGL | |
| hu 4-1BBL (80- | DPAGLLDLRQGMFAQLVAQNVLLIDGPLSWYSDPGLAGVSLTGGL | 34 |
| 248) | SYKEDTKELVVAKAGVYYVFFQLELRRVVAGEGSGSVSLALHLQP | |
| | LRSAAGAAALALTVDLPPASSEARNSAFGFQGRLLHLSAGQRLGV | |
| | HLHTEARARHAWQLTQGATVLGLFRVTPEIPAGL | |
| hu 4-1BBL (52- | PWAVSGARASPGSAASPRLREGPELSPDDPAGLLDLRQGMFAQLV | 35 |
| 248) | AQNVLLIDGPLSWYSDPGLAGVSLTGGLSYKEDTKELVVAKAGVY | |
| | YVFFQLELRRVVAGEGSGSVSLALHLQPLRSAAGAAALALTVDLP | |
| | PASSEARNSAFGFQGRLLHLSAGQRLGVHLHTEARARHAWQLTQG | |
| | ATVLGLFRVTPEIPAGL | |
| FAP HCDR1 | SYAMS | 36 |
| FAP HCDR2 | AIIGSGASTYYADSVKG | 37 |
| FAP HCDR3 | GWFGGFNY | 38 |
| FAP LCDR1 | RASQSVTSSYLA | 39 |
| FAP LCDR2 | VGSRRAT | 40 |
| FAP LCDR3 | QQGIMLPPT | 41 |
| FAP VH | EVQLLESGGGLVQPGGSLRLSCAASGFTFSSYAMSWVRQAPGKGL | 42 |
| | EWVSAIIGSGASTYYADSVKGRFTISRDNSKNTLYLQMNSLRAED | |
| | TAVYYCAKGWFGGFNYWGQGTLVTVSS | |
| FAP VL | EIVLTQSPGTLSLSPGERATLSCRASQSVTSSYLAWYQQKPGQAP | 43 |
| | RLLINVGSRRATGIPDRESGSGSGTDETLTISRLEPEDFAVYYCQ | |
| | QGIMLPPTFGQGTKVEIK | |
| hu 4-1BBL (71- | REGPELSPDDPAGLLDLRQGMFAQLVAQNVLLIDGPLSWYSDPGL | 44 |
| 248)-(G₄S)₂-hu 4- | AGVSLTGGLSYKEDTKELVVAKAGVYYVFFQLELRRVVAGEGSGS | |
| 1BBL (71-248) | VSLALHLQPLRSAAGAAALALTVDLPPASSEARNSAFGFQGRLLH | |
| | LSAGQRLGVHLHTEARARHAWQLTQGATVLGLFRVTPEIPAGLGG | |
| | GGSGGGGSREGPELSPDDPAGLLDLRQGMFAQLVAQNVLLIDGPL | |
| | SWYSDPGLAGVSLTGGLSYKEDTKELVVAKAGVYYVFFQLELRRV | |
| | VAGEGSGSVSLALHLQPLRSAAGAAALALTVDLPPASSEARNSAF | |
| | GFQGRLLHLSAGQRLGVHLHTEARARHAWQLTQGATVLGLFRVTP | |
| | EIPAGL | |
| hu 4-1BBL (71- | REGPELSPDDPAGLLDLRQGMFAQLVAQNVLLIDGPLSWYSDPGL | 45 |
| 248)-(G₄S)₂-hu 4- | AGVSLTGGLSYKEDTKELVVAKAGVYYVFFQLELRRVVAGEGSGS | |
| 1BBL (71-248)- | VSLALHLQPLRSAAGAAALALTVDLPPASSEARNSAFGFQGRLLH | |
| CL(RK)-Fc(knob, | LSAGQRLGVHLHTEARARHAWQLTQGATVLGLFRVTPEIPAGLGG | |
| PGLALA) | GGSGGGGSREGPELSPDDPAGLLDLRQGMFAQLVAQNVLLIDGPL | |
| | SWYSDPGLAGVSLTGGLSYKEDTKELVVAKAGVYYVFFQLELRRV | |
| | VAGEGSGSVSLALHLQPLRSAAGAAALALTVDLPPASSEARNSAF | |
| | GFQGRLLHLSAGQRLGVHLHTEARARHAWQLTQGATVLGLERVTP | |
| | EIPAGLGGGGSGGGGSRTVAAPSVFIFPPSDRKLKSGTASVVCLL | |
| | NNFYPREAKVQWKVDNALQSGNSQESVTEQDSKDSTYSLSSTLTL | |
| | SKADYEKHKVYACEVTHQGLSSPVTKSENRGECDKTHTCPPCPAP | |
| | EAAGGPSVFLFPPKPKDTLMISRTPEVTCVVVDVSHEDPEVKENW | |
| | YVDGVEVHNAKTKPREEQYNSTYRVVSVLTVLHQDWLNGKEYKCK | |
| | VSNKALGAPIEKTISKAKGQPREPQVYTLPPCRDELTKNQVSLWC | |
| | LVKGFYPSDIAVEWESNGQPENNYKTTPPVLDSDGSFFLYSKLTV | |
| | DKSRWQQGNVFSCSVMHEALHNHYTQKSLSLSP | |
| hu 4-1BBL (71- | REGPELSPDDPAGLLDLRQGMFAQLVAQNVLLIDGPLSWYSDPGL | 46 |
| 248)-CH1(EE) | AGVSLTGGLSYKEDTKELVVAKAGVYYVFFQLELRRVVAGEGSGS | |
| | VSLALHLQPLRSAAGAAALALTVDLPPASSEARNSAFGFQGRLLH | |
| | LSAGQRLGVHLHTEARARHAWQLTQGATVLGLFRVTPEIPAGLGG | |
| | GGSGGGGSASTKGPSVFPLAPSSKSTSGGTAALGCLVEDYFPEPV | |
| | TVSWNSGALTSGVHTFPAVLQSSGLYSLSSVVTVPSSSLGTQTYI | |
| | CNVNHKPSNTKVDEKVEPKSC | |
| FAP VH-CH1- | EVQLLESGGGLVQPGGSLRLSCAASGFTFSSYAMSWVRQAPGKGL | 47 |
| Fc(hole, PGLALA) | EWVSAIIGSGASTYYADSVKGRFTISRDNSKNTLYLQMNSLRAED | |
| | TAVYYCAKGWFGGFNYWGQGTLVTVSSASTKGPSVFPLAPSSKST | |
| | SGGTAALGCLVKDYFPEPVTVSWNSGALTSGVHTFPAVLQSSGLY | |
| | SLSSVVTVPSSSLGTQTYICNVNHKPSNTKVDKKVEPKSCDKTHT | |
| | CPPCPAPEAAGGPSVFLFPPKPKDTLMISRTPEVTCVVVDVSHED | |
| | PEVKFNWYVDGVEVHNAKTKPREEQYNSTYRVVSVLTVLHQDWLN | |
| | GKEYKCKVSNKALGAPIEKTISKAKGQPREPQVCTLPPSRDELTK | |
| | NQVSLSCAVKGFYPSDIAVEWESNGQPENNYKTTPPVLDSDGSFF | |
| | LVSKLTVDKSRWQQGNVFSCSVMHEALHNHYTQKSLSLSP | |
| FAP VL-CL | EIVLTQSPGTLSLSPGERATLSCRASQSVTSSYLAWYQQKPGQAP | 48 |
| | RLLINVGSRRATGIPDRESGSGSGTDFTLTISRLEPEDFAVYYCQ | |
| | QGIMLPPTFGQGTKVEIKRTVAAPSVFIFPPSDEQLKSGTASVVC | |
| | LLNNFYPREAKVQWKVDNALQSGNSQESVTEQDSKDSTYSLSSTL | |
| | TLSKADYEKHKVYACEVTHQGLSSPVTKSENRGEC | |

### EXAMPLES

The following are examples of methods and compositions of the invention. It is understood that various other aspects may be practiced, given the general description provided above.

### Example 1—Continuous Live-cell Imaging to Monitor MAGE-A4 TCB +FAP-4-1-BBL-mediated Tumor Cell Lysis

To monitor tumor cell lysis induced by a combination of MAGE-A4 TCB+FAP-4-1-BBL in a kinetic manner over several days, continuous live-cell imaging was performed with two different MAGE-A4+/HLA-A*02:01+cell lines (SCaBER, a cell line derived from squamous cell carcinoma of the human urinary bladder (O'Toole et al., Int. J. Cancer 17: 707-714 (1976)) and C-33a, a cell line derived from human cervical carcinoma (Auersperg, J. Natl. Cancer Inst. 32:135-163 (1964))). Cells were incubated with human PBMCs in the presence of irradiated NIH/3T3-huFAP fibroblast cells (NIH/3T3 cells stably expressing human FAP, irradiated to prevent growth) and treated with a combination of 2 nM FAP-4-1-BBL and various concentrations (50 nM-1 pM) of MAGE-A4 TCB. As controls, tumor cells were incubated with PBMCs in the absence of a treatment (=untreated), with 2 nM of FAP-4-1-BBL only, or with various concentrations of MAGE-A4 TCB only.

One day before the assay setup, healthy donor PBMCs (Lonza) were thawed and resuspended in RPMI1640 medium containing 10% FCS, and 1% GlutaMAX. Cells were maintained in the incubator at 37° C. and 5% CO₂ overnight.

In parallel, target tumor cell lines were collected, and their viability and cell number was assessed using a ViCell XR cell counter (Beckman Coulter). After centrifugation (350×g, 4° C., 5 minutes), the cell number was adjusted to 8×10⁴ cells/mL (SCaBER H2BRFP, i.e. transduced with a lentivirus (Essen Bioscience, #4476) to stably express a nuclear red fluorescent protein), 24×10⁴ cells/mL (C-33a H2BRFP), or 16×10⁴ cells/mL (NIH/3T3-huFAP clone 19 irradiated) in RPMI1640 containing 10% FCS, and 1% GlutaMAX. 62.5 μL of the cell suspension were plated to obtain a final cell density of 5000, 15,000, or 10,000 cells/well, respectively. All edge wells were filled with assay medium to avoid evaporation of liquid in assay wells. The assay plates containing the target cells were incubated at 37° C. and 5% CO₂ overnight.

On the day of the assay, 62.5 μL of MAGE-A4 TCB was added to the target cells to obtain a final concentration of 50 nM-1 μM of per well. 2 nM of FAP-4-1BBL or assay medium for MAGE-A4 TCB only treatments were added. For “no antibody” controls, assay medium was added.

Previously thawed PBMCs (see above) were taken from the incubator, counted with the ViCell XR cell counter (Beckman Coulter) and centrifuged (350×g, 5 minutes). PBMCs were resuspended at 4×10⁵ cells/mL (for SCaBER H2BRFP) or 1.2×10⁶ cells/mL (for C33a H2BRFP) in assay medium. The respective PBMC solutions were plated per well to obtain an effector-to-target (E:T) ratio of 5:1 in a final volume of 250 μL.

After centrifugation at 200×g for 1 minute, plates were placed in the incubator of the Incucyte S3 Live-Cell Analysis System (Essen Bioscience, Ltd.) at 37° C. and 5% CO₂, and scanning started 1 hour later (Time 0 hours). Plates were scanned every 3 hours for a total of 120 hours by taking four pictures per well (phase contrast, red channel [i.e., target cells]). The average signal at Time 0 hours was calculated from three replicates in each condition. This value was subtracted from each value of the same condition at later timepoints, yielding the normalized values.

After 48 hours, 25 μl of the supernatant from each well were transferred to fresh plates and frozen at −80° C. for Luminex cytokine analysis (see Example 0).

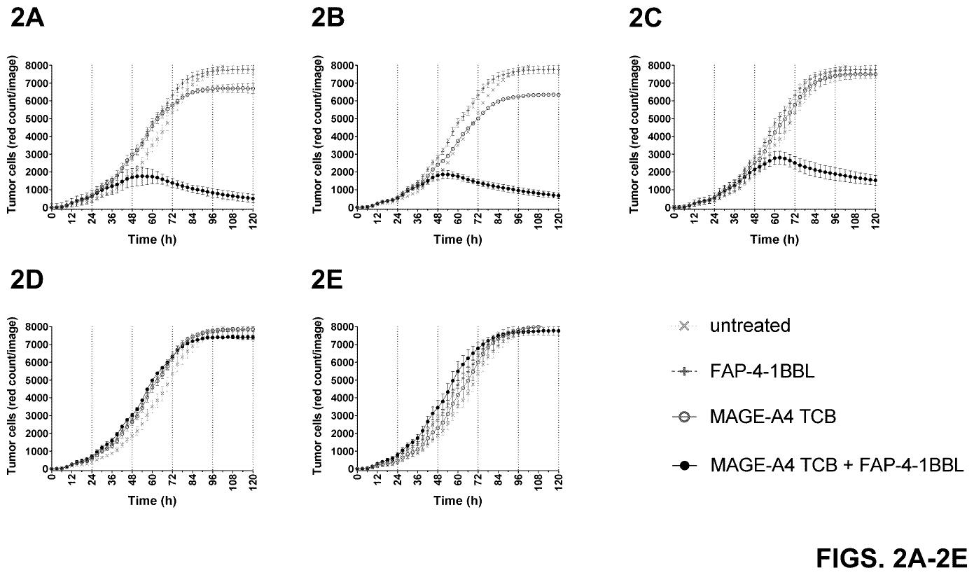

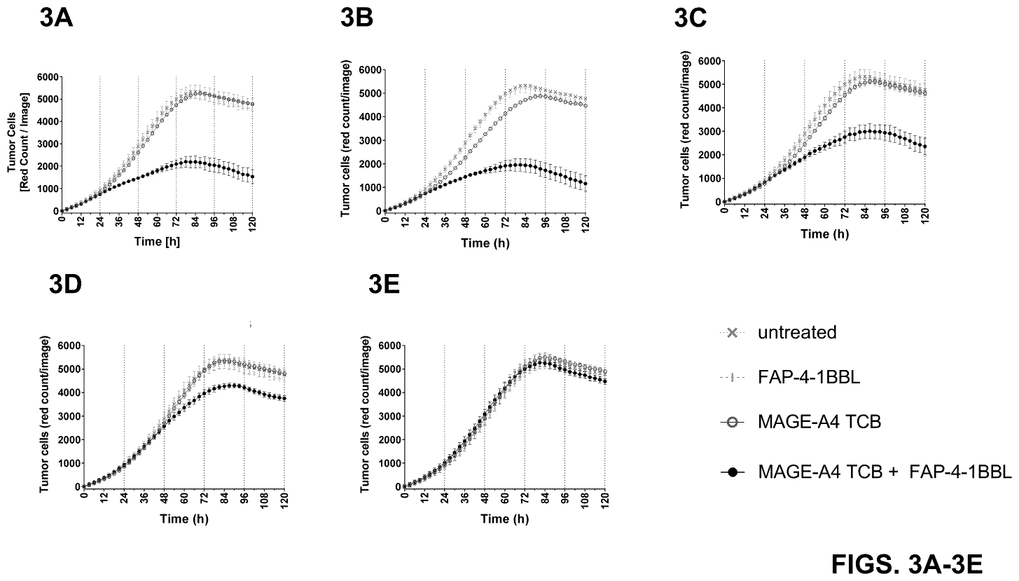

The results are shown in FIGS. 2A-2E and FIGS. 3A-3E. The normalized values of the number of tumor cells per image (i.e., red signal per image) were plotted.

As shown in FIGS. 2A-2E and FIGS. 3A-3E, a combination of MAGE-A4 TCB with the targeted co-stimulator FAP-4-1-BBL leads to a strong inhibition in tumor cell growth as monitored by continuous live cell imaging. In contrast, MAGE-A4 TCB does not or only very weakly (at high concentrations) induce tumor growth inhibition when used as single treatment, which shows that the non-responsiveness of SCaBER and C-33a cells towards MAGE-A4 TCB monotherapy can be overcome by a combination therapy with FAP-4-1-BBL.

Example 2—Luminex immunoassays to quantify cytokine release induced by a combination of MAGE-A4 TCB +FAP-4-1-BBL

In addition to continuous live-cell imaging, the pharmacological activity of the combination of MAGE-A4 TCB+FAP-4-1-BBL was further evaluated by quantifying the levels of cytokines and cytotoxic granules (Granzyme B) released into the cell supernatant by activated T cells. Cytokines released into the cell culture supernatant were quantified via Luminex immunoassays by using a Human Custom ProcartaPlex 7-plex kit (Thermo Fisher Scientific #PPX-07-MXFVK4Y).

On the day of the assay, frozen cell supernatants from tumor cell lysis experiments were thawed on ice. Triplicates were mixed well by vortexing and pooled into one well. Samples were used 1:3 diluted in assay medium (RPMI1640, 10% FBS, 1% GlutaMAX).

The standard vials (provided in the kit) were centrifuged at 2000×g for 10 seconds and reconstituted by adding assay medium according to the manufacturer's instructions (ThermoFisher Scientific). A 3-fold standard dilution series in assay medium and a blank (medium only) sample were added according to the plate layout into the suspension plate.

Magnetic beads were vortexed for 30 seconds, diluted 1:2 in assay medium, and protected from light (by aluminum foil).

Filter plates were pre-wetted for 5 minutes by adding 100 μL/well of PBS and dried on a vacuum manifold.

The beads solution was vortexed for 30 seconds, and 50 μL/well were added to the filter assay plates. After washing twice with 100 μL/well wash buffer, 50 μL/well of samples, standards, or blanks were added, and plates were incubated at room temperature in the dark on a plate shaker (500 rpm) for >60 min.

Filter plates were washed twice with 100 μl/well wash buffer. Then, 1×detection antibody mixture was diluted 1:2 in PBS or Universal Assay Buffer, and 25 μl were added to each well and incubated at room temperature in the dark on a plate shaker (500 rpm) for 30 minutes.

After washing twice with 100 μL/well wash buffer, 50 μL/well of SA-RPE solution (diluted 1:2 in PBS or Universal Assay Buffer) were added to each well. Plates were incubated at room temperature in the dark on a plate shaker (500 rpm) for 30 minutes. After washing twice with 100 μL/well wash buffer, 100 μL/well of reading buffer were added to each well, and plates were incubated for another 5 minutes at the same conditions.

Plates were read on a BioPlex® (BioRad) plate reader, and the obtained signals were quantitated automatically by Bio-Plex Manager 6.1.1 software.

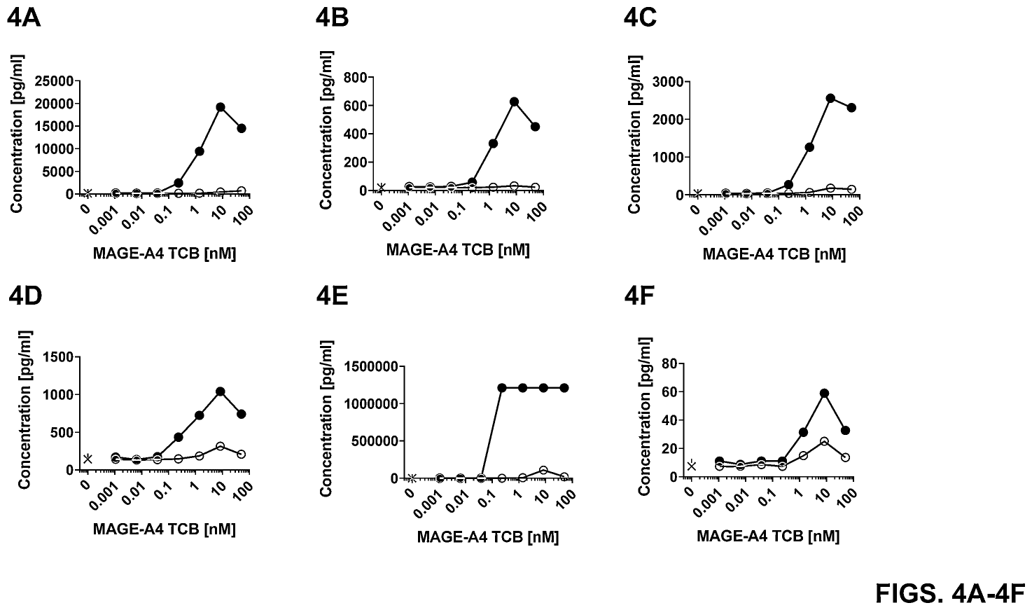

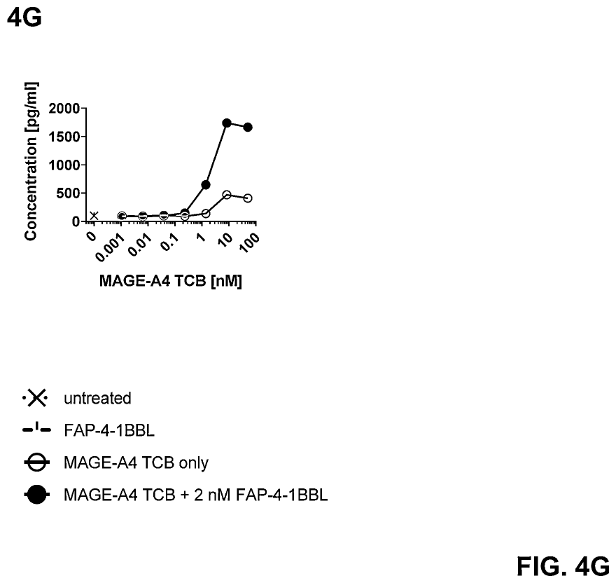

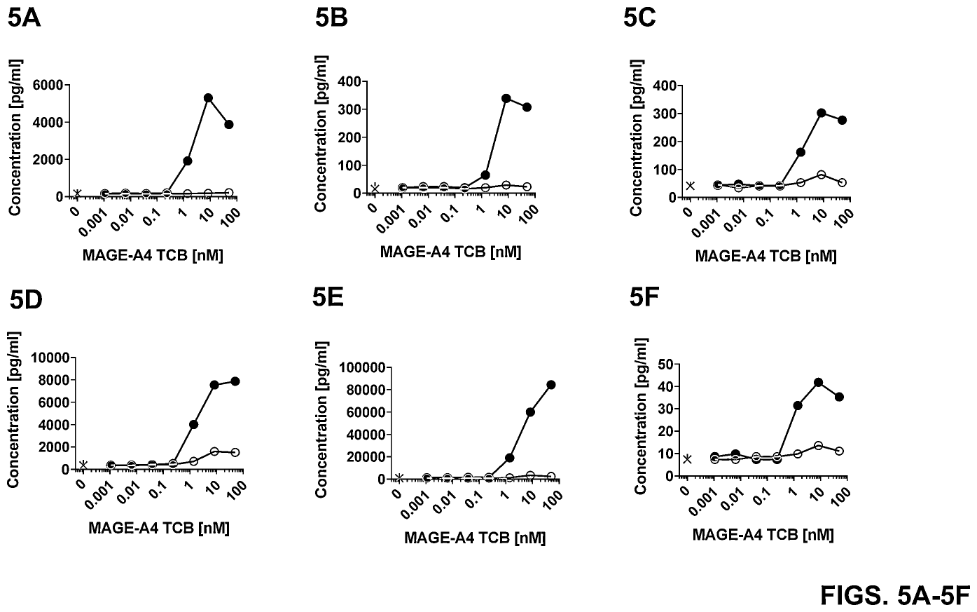

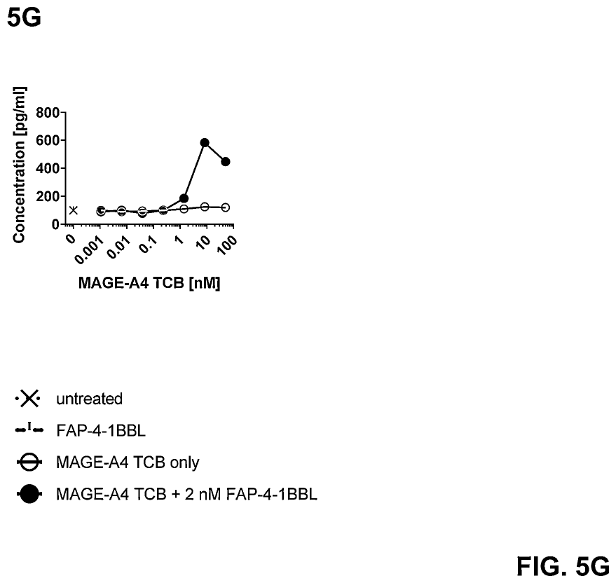

The results are shown in FIGS. 4A-4G and FIGS. 5A-5G. Cytokine concentration values were transferred to GraphPad Prism and plotted. Each cytokine concentration value was corrected with a baseline subtraction. Baseline refers to cytokine concentrations that accumulated in the supernatants of wells co-cultured with target cells and PBMCs without any treatment molecules. A sigmoidal dose-response, four-parameter logistic equation, and least squares (ordinary) fit analysis was applied to the baseline-subtracted values to produce a dose-response curve fit.

In line with the results obtained for the continuous live cell imaging, a dose-dependent cytokine secretion could be observed for MAGE-A4 TCB+FAP-4-1BBL-activated T cells (FIGS. 4A-4G and FIGS. 5A-5G) whereas no or only very low cytokine release could be observed for MAGE-A4 TCB monotherapy.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A HLA-A2/MAGE-A4×CD3 bispecific antibody for use in the treatment of a cancer in an individual, wherein the treatment comprises administration of the HLA-A2/MAGE-A4×CD3 bispecific antibody in combination with a 4-1BB (CD137) agonist.

2. A 4-1BB (CD137) agonist for use in the treatment of a cancer in an individual, wherein the treatment comprises administration of the 4-1BB (CD137) agonist in combination with a HLA-A2/MAGE-A4×CD3 bispecific antibody.

3. Use of a HLA-A2/MAGE-A4×CD3 bispecific antibody in the manufacture of a medicament for the treatment of cancer in an individual, wherein the treatment comprises administration of the HLA-A2/MAGE-A4×CD3 bispecific antibody in combination with a 4-1BB (CD137) agonist.

4. Use of a 4-1BB (CD137) agonist in the manufacture of a medicament for the treatment of cancer in an individual, wherein the treatment comprises administration of the 4-1BB (CD137) agonist in combination with a HLA-A2/MAGE-A4×CD3 bispecific antibody.

5. A method for treating cancer in an individual comprising administering to the individual a HLA-A2/MAGE-A4×CD3 bispecific antibody and a 4-1BB (CD137) agonist.

6. A kit comprising a first medicament comprising a HLA-A2/MAGE-A4×CD3 bispecific antibody and a second medicament comprising a 4-1BB (CD137) agonist, and optionally further comprising a package insert comprising instructions for administration of the first medicament in combination with the second medicament for treating cancer in an individual.

7. The HLA-A2/MAGE-A4×CD3 bispecific antibody for use, the 4-1BBL (CD137) agonist for use, the use, the method or the kit of any one of the preceding claims, wherein the HLA-A2/MAGE-A4×CD3 bispecific antibody comprises
(i) a first antigen binding moiety that specifically binds to CD3 and comprises a heavy chain variable region comprising the heavy chain CDR (HCDR) 1 of SEQ ID NO: 1, the HCDR2 of SEQ ID NO: 2, and the HCDR3 of SEQ ID NO: 3; and a light chain variable region comprising the light chain CDR (LCDR) 1 of SEQ ID NO: 4, the LCDR2 of SEQ ID NO: 5 and the LCDR3 of SEQ ID NO: 6; and
(ii) a second antigen binding moiety that specifically binds to HLA-A2/MAGE-A4 and comprises a heavy chain variable region comprising the heavy chain CDR (HCDR) 1 of SEQ ID NO: 9, the HCDR2 of SEQ ID NO: 10, and the HCDR3 of SEQ ID NO: 11; and a light chain variable region comprising the light chain CDR (LCDR) 1 of SEQ ID NO: 12, the LCDR2 of SEQ ID NO: 13 and the LCDR3 of SEQ ID NO: 14.

8. The HLA-A2/MAGE-A4×CD3 bispecific antibody for use, the use, the 4-1BBL (CD137) agonist for use, the use, the method or the kit of any one of the preceding claims, wherein the HLA-A2/MAGE-A4×CD3 bispecific antibody comprises a third antigen binding moiety that specifically binds to HLA-A2/MAGE-A4 and/or an Fc domain composed of a first and a second subunit.

9. The HLA-A2/MAGE-A4×CD3 bispecific antibody for use, the 4-1BBL (CD137) agonist for use, the use, the use, the method or the kit of any one of the preceding claims, wherein the HLA-A2/MAGE-A4×CD3 bispecific antibody comprises
(i) a first antigen binding moiety that specifically binds to CD3, comprising a heavy chain variable region comprising the heavy chain CDR (HCDR) 1 of SEQ ID NO: 1, the HCDR2 of SEQ ID NO: 2, and the HCDR3 of SEQ ID NO: 3; and a light chain variable region comprising the light chain CDR (LCDR) 1 of SEQ ID NO: 4, the LCDR2 of SEQ ID NO: 5 and the LCDR3 of SEQ ID NO: 6, wherein the first antigen binding moiety is a crossover Fab molecule wherein either the variable or the constant regions of the Fab light chain and the Fab heavy chain are exchanged;
(ii) a second and a third antigen binding moiety that specifically bind to HLA-A2/MAGE-A4, comprising a heavy chain variable region comprising the heavy chain CDR (HCDR) 1 of SEQ ID NO: 9, the HCDR2 of SEQ ID NO: 10, and the HCDR3 of SEQ ID NO: 11; and a light chain variable region comprising the light chain CDR (LCDR) 1 of SEQ ID NO: 12, the LCDR2 of SEQ ID NO: 13 and the LCDR3 of SEQ ID NO: 14, wherein the second and third antigen binding moiety are each a Fab molecule, particularly a conventional Fab molecule;
(iii) an Fc domain composed of a first and a second subunit,
wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain.

10. The HLA-A2/MAGE-A4×CD3 bispecific antibody for use, the use, the 4-1BBL (CD137) agonist for use, the use, the method or the kit of any one of the preceding claims, wherein the first antigen binding moiety of the HLA-A2/MAGE-A4×CD3 bispecific antibody comprises a heavy chain variable region sequence that is at least about 95%, 96%, 97%, 98%, 99% or 100% identical to the amino acid sequence of SEQ ID NO: 7 and a light chain variable region sequence that is at least about 95%, 96%, 97%, 98%, 99% or 100% identical to the amino acid sequence of SEQ ID NO: 8, and/or the second and (where present) third antigen binding moiety of the HLA-A2/MAGE-A4×CD3 bispecific antibody comprise a heavy chain variable region sequence that is at least about 95%, 96%, 97%, 98%, 99% or 100% identical to the amino acid sequence of SEQ ID NO: 15 and a light chain variable region sequence that is at least about 95%, 96%, 97%, 98%, 99% or 100% identical to the amino acid sequence of SEQ ID NO: 16.

11. The HLA-A2/MAGE-A4×CD3 bispecific antibody for use, the 4-1BBL (CD137) agonist for use, the use, the use, the method or the kit of any one of the preceding claims, wherein the first antigen binding moiety of the HLA-A2/MAGE-A4×CD3 bispecific antibody is a crossover Fab molecule wherein the variable regions of the Fab light chain and the Fab heavy chain are exchanged, and wherein the second and (where present) third antigen binding moiety of the HLA-A2/MAGE-A4×CD3 bispecific antibody is a conventional Fab molecule wherein in the constant domain CL the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat) and in the constant domain CH1 the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

12. The HLA-A2/MAGE-A4×CD3 bispecific antibody for use, the 4-1BBL (CD137) agonist for use, the use, the use, the method or the kit of any one of the preceding claims, wherein the Fc domain of the HLA-A2/MAGE-A4×CD3 bispecific antibody comprises a modification promoting the association of the first and the second subunit of the Fc domain, and/or the Fc domain comprises one or more amino acid substitution that reduces binding to an Fc receptor and/or effector function.

13. The HLA-A2/MAGE-A4×CD3 bispecific antibody for use, the 4-1BBL (CD137) agonist for use, the use, the use, the method or the kit of any one of the preceding claims, wherein the 4-1BB (CD137) agonist comprises an ectodomain of 4-1BBL or fragment thereof, particularly three ectodomains of 4-1BBL or fragments thereof.

14. The HLA-A2/MAGE-A4×CD3 bispecific antibody for use, the 4-1BBL (CD137) agonist for use, the use, the use, the method or the kit of any one of the preceding claims, wherein the ectodomain of 4-1BBL or fragment thereof comprises an amino acid sequence that is at least about 95%, 96%, 97%, 98%, 99% or 100% identical to an amino acid sequence selected from the group consisting of SEQ ID NO: 28, SEQ ID NO: 29, SEQ ID NO: 30, SEQ ID NO: 31, SEQ ID NO: 32, SEQ ID NO:
33, SEQ ID NO: 34 and SEQ ID NO: 35, particularly the amino acid sequence of SEQ ID NO: 32.

15. The HLA-A2/MAGE-A4×CD3 bispecific antibody for use, the 4-1BBL (CD137) agonist for use, the use, the use, the method or the kit of any one of the preceding claims, wherein the 4-1BB (CD137) agonist comprises an antigen binding moiety that specifically binds to a tumor-associated antigen, particularly Fibroblast Activation Protein (FAP).

16. The HLA-A2/MAGE-A4×CD3 bispecific antibody for use, the 4-1BBL (CD137) agonist for use, the use, the use, the method or the kit of claim 15, wherein the antigen binding moiety that specifically binds to FAP comprises a heavy chain variable region (VH) comprising the heavy chain CDR (HCDR) 1 of SEQ ID NO: 36, the HCDR2 of SEQ ID NO: 37, and the HCDR3 of SEQ ID NO: 38; and a light chain variable region comprising the light chain CDR (LCDR) 1 of SEQ ID NO: 39, the LCDR2 of SEQ ID NO: 40 and the LCDR3 of SEQ ID NO: 41.

17. The HLA-A2/MAGE-A4×CD3 bispecific antibody for use, the 4-1BBL (CD137) agonist for use, the use, the use, the method or the kit of claim 15 or 16, wherein the antigen binding moiety that specifically binds to FAP comprises a heavy chain variable region sequence that is at least about 95%, 96%, 97%, 98%, 99% or 100% identical to the amino acid sequence of SEQ ID NO: 42 and a light chain variable region sequence that is at least about 95%, 96%, 97%, 98%, 99% or 100% identical to the amino acid sequence of SEQ ID NO: 43.

18. The HLA-A2/MAGE-A4×CD3 bispecific antibody for use, the 4-1BBL (CD137) agonist for use, the use, the use, the method or the kit of any one of the preceding claims, wherein the 4-1BB (CD137) agonist comprises an Fc domain composed of a first and a second subunit.

19. The HLA-A2/MAGE-A4×CD3 bispecific antibody for use, the 4-1BBL (CD137) agonist for use, the use, the use, the method or the kit of claim 18, wherein the Fc domain of 4-1BB (CD137) agonist comprises a modification promoting the association of the first and the second subunit of the Fc domain, and/or the Fc domain comprises one or more amino acid substitution that reduces binding to an Fc receptor and/or effector function.

20. The HLA-A2/MAGE-A4×CD3 bispecific antibody for use, the 4-1BBL (CD137) agonist for use, the use, the use, the method or the kit of any one of the preceding claims, wherein the 4-1BBL (CD137) agonist is an antigen binding molecule comprising
(i) a first, a second and a third ectodomain of 4-1BBL or fragment thereof;
(ii) an antigen binding moiety that specifically binds to FAP, wherein the antigen binding moiety is a Fab molecule;
(iii) an Fc domain composed of a first and a second subunit;
(iv) a CL domain and a CH1 domain;
wherein the antigen binding molecule is composed of
(a) a first polypeptide, comprising the (a1) first ectodomain of 4-1BBL or fragment thereof, fused at its C-terminus to the N-terminus of the second ectodomain of 4-1BBL or fragment thereof, (a2) the second ectodomain of 4-1BBL or fragment thereof, fused at its C-terminus to the N-terminus of the CL domain, (a3) the CL domain, fused at its C-terminus to the N-terminus of one of the subunits (e.g. the first subunit) of the Fc domain, and (a4) one of the subunits (e.g. the first subunit) of the Fc domain;
(b) a second polypeptide, comprising (b1) the third ectodomain of 4-1BBL or fragment thereof, fused at its C-terminus to the N-terminus of the CH1 domain, and (b2) the CH1 domain;
(c) a third polypeptide, comprising (c1) the heavy chain of the Fab molecule, fused at its C-terminus to the N-terminus of the other one of the subunits (e.g. the second subunit) of the Fc domain, and (c2) the other one of the subunits (e.g. the second subunit) of the Fc domain; and
(d) a fourth polypeptide, comprising the light chain of the Fab molecule.

21. The HLA-A2/MAGE-A4×CD3 bispecific antibody for use, the 4-1BBL (CD137) agonist for use, the use, the use, the method or the kit of claim 20, wherein in the CL domain of the first polypeptide the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the CH1 domain of the second polypeptide the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

22. The HLA-A2/MAGE-A4×CD3 bispecific antibody for use, the 4-1BBL (CD137) agonist for use, the use, the use, the method or the kit of claim 20 or 21, wherein the first polypeptide comprises an amino acid sequence that is at least about 95%, 96%, 97%, 98%, 99% or 100% identical to the amino acid sequence of of SEQ ID NO: 45, the second polypeptide comprises an amino acid sequence that is at least about 95%, 96%, 97%, 98%, 99% or 100% identical to the amino acid sequence of of SEQ ID NO: 46, the third polypeptide comprises an amino acid sequence that is at least about 95%, 96%, 97%, 98%, 99% or 100% identical to the amino acid sequence of of SEQ ID NO: 47, and the fourth polypeptide comprises an amino acid sequence that is at least about 95%, 96%, 97%, 98%, 99% or 100% identical to the amino acid sequence of of SEQ ID NO: 48.

23. The HLA-A2/MAGE-A4×CD3 bispecific antibody for use, the 4-1BBL (CD137) agonist for use, the use, the use, the method or the kit of any one of claims 1 to 12, wherein the 4-1BB (CD137) agonist is an anti-4-1BB antibody, particularly an anti-FAP/anti-4-1BB bispecific antibody.

24. The HLA-A2/MAGE-A4×CD3 bispecific antibody for use, the 4-1BBL (CD137) agonist for use, the use, the use, the method or the kit of any one of the preceding claims, wherein the cancer is a MAGE-A4-positive cancer.

25. The HLA-A2/MAGE-A4×CD3 bispecific antibody for use, the 4-1BBL (CD137) agonist for use, the use, the use, the method or the kit of any one of the preceding claims, wherein the cancer is a cancer selected from the group consisting of lung cancer, head and neck cancer, bladder cancer, esophageal cancer, skin cancer, soft tissue cancer, gastric cancer, cervical cancer and ovarian cancer.

26. The invention as described hereinbefore.


SEQUENCE LISTING (48 sequences)

SEQ ID NO: 1 - protein, 5 aa, synthetic construct
GYTMN

SEQ ID NO: 2 - protein, 17 aa, synthetic construct
LINPYKGVSTYNQKFKD

SEQ ID NO: 3 - protein, 13 aa, synthetic construct
SGYYGDSDWYFDV

SEQ ID NO: 4 - protein, 11 aa, synthetic construct
RASQDIRNYLN

SEQ ID NO: 5 - protein, 7 aa, synthetic construct
YTSRLES

SEQ ID NO: 6 - protein, 9 aa, synthetic construct
QQGNTLPWT

SEQ ID NO: 7 - protein, 122 aa, synthetic construct [sequence omitted]

SEQ ID NO: 8 - protein, 107 aa, synthetic construct [sequence omitted]

SEQ ID NO: 9 - protein, 4 aa, synthetic construct
KAMS

SEQ ID NO: 10 - protein, 17 aa, synthetic construct
SISPSGGSTYYNDNVLG

SEQ ID NO: 11 - protein, 7 aa, synthetic construct
DVGFFDE

SEQ ID NO: 12 - protein, 11 aa, synthetic construct
RASQSISSYLA

SEQ ID NO: 13 - protein, 7 aa, synthetic construct
DASIRDI

SEQ ID NO: 14 - protein, 9 aa, synthetic construct
QQYSSYPYT

SEQ ID NO: 15 - protein, 114 aa, synthetic construct
AQLVESGGGLVQPGGSLRLSCAASAYFSFKAMSWVRQAPGKGLEWVGSISPSGGSTYYNDNVLGRFTISRDNSKNTLYLQMNSLRAEDTAVYYCAKDVGFFDEWGQGTLVTVSS

SEQ ID NO: 16 - protein, 107 aa, synthetic construct
DIQMTQSPSSLSASVGDRVTITCRASQSISSYLAWYQQKPGKAPKLLIYDASIRDIGVPSRFSGSGSGTDFTLTISSLQPEDFATYYCQQYSSYPYTFGQGTKLEIK

SEQ ID NO: 17 - protein, 214 aa, synthetic construct
DIQMTQSPSSLSASVGDRVTITCRASQSISSYLAWYQQKPGKAPKLLIYDASIRDIGVPSRFSGSGSGTDFTLTISSLQPEDFATYYCQQYSSYPYTFGQGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 442 aa, synthetic construct
AQLVESGGGLVQPGGSLRLSCAASAYFSFKAMSWVRQAPGKGLEWVGSISPSGGSTYYNDNVLGRFTISRDNSKNTLYLQMNSLRAEDTAVYYCAKDVGFFDEWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 19 - protein, 229 aa, synthetic construct
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 665 aa, synthetic construct
AQLVESGGGLVQPGGSLRLSCAASAYFSFKAMSWVRQAPGKGLEWVGSISPSGGSTYYNDNVLGRFTISRDNSKNTLYLQMNSLRAEDTAVYYCAKDVGFFDEWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 21 - protein, 317 aa, Homo sapiens
MSSEQKSQHCKPEEGVEAQEEALGLVGAQAPTTEEQEAAVSSSSPLVPGTLEEVPAAESAGPPQSPQGASALPTTISFTCWRQPNEGSSSQEEEGPSTSPDAESLFREALSNKVDELAHFLLRKYRAKELVTKAEMLERVIKNYKRCFPVIFGKASESLKMIFGIDVKEVDPASNTYTLVTCLGLSYDGLLGNNQIFPKTGLLIIVLGTIAMEGDSASEEEIWEELGVMGVYDGREHTVYGEPRKLLTQDWVQENYLEYRQVPGSNPARYEFLWGPRALAETSYVKVLEHVVRVNARVRIAYPSLREAALLEEEEGV

SEQ ID NO: 22 - protein, 10 aa, Homo sapiens
GVYDGREHTV

SEQ ID NO: 23 - protein, 275 aa, Homo sapiens [sequence omitted]

SEQ ID NO: 24 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 25 - protein, 198 aa, Macaca fascicularis
MQSGTRWRVLGLCLLSIGVWGQDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 26 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 27 - protein, 205 aa, Homo sapiens
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 28 - protein, 184 aa, Homo sapiens
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 29 - protein, 170 aa, Homo sapiens
LDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 30 - protein, 175 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 31 - protein, 203 aa, Homo sapiens
PWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 32 - protein, 178 aa, Homo sapiens
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 33 - protein, 164 aa, Homo sapiens
LDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 34 - protein, 169 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 35 - protein, 197 aa, Homo sapiens
PWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 36 - protein, 5 aa, synthetic construct
SYAMS

SEQ ID NO: 37 - protein, 17 aa, synthetic construct
AIIGSGASTYYADSVKG

SEQ ID NO: 38 - protein, 8 aa, synthetic construct
GWFGGFNY

SEQ ID NO: 39 - protein, 12 aa, synthetic construct
RASQSVTSSYLA

SEQ ID NO: 40 - protein, 7 aa, synthetic construct
VGSRRAT

SEQ ID NO: 41 - protein, 9 aa, synthetic construct
QQGIMLPPT

SEQ ID NO: 42 - protein, 117 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 43 - protein, 108 aa, synthetic construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 44 - protein, 366 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 45 - protein, 708 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 46 - protein, 291 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSC

SEQ ID NO: 47 - protein, 445 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 48 - protein, 215 aa, synthetic construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC